(19) United States
(12) Patent Application Publication
(10) Pub. No.: US 2008/0161422 A1
(43) Pub. Date: Jul. 3, 2008

(54) Methods and Compositions for the Treatment of Lipid-Associated Disorders

(76) Inventors: Martha Kanemitsu-Parks, Del Mar, CA (US); Jeremy G. Richman, San Diego, CA (US); Dominique Maciejewski-Lenoir, San Diego, CA (US); Daniel T. Connolly, Solana Beach, CA (US)

Correspondence Address: FISH & RICHARDSON P.C., P.O. BOX 1022, MINNEAPOLIS, MN, 55440-1022, US

(21) Appl. No.: 11/884,675

(22) PCT Filed: Feb. 18, 2006
(86) PCT No.: PCT/US2006/005450

§ 371 Date: Oct. 12, 2007

Related U.S. Application Data
(60) Provisional application No. 60/654,684, filed on Feb. 18, 2005.

(51) Int. Cl. A61K 35/00; C12Q 1/48; G01N 33/566

(52) U.S. Cl. 514/789; 435/15; 435/7.92

(57) ABSTRACT

The invention provides a method of identifying a niacin receptor modulator with reduced flushing effect compared to niacin or a niacin analog, comprising determining the MAP kinase activity of said modulator, wherein a decrease in MAP kinase activity induced by said modulator compared to MAP kinase activity induced by niacin or a niacin analog indicates that said modulator has reduced flushing effect when compared to niacin or a niacin analog.

### FIELD OF THE INVENTION

The present invention relates generally to treatment of lipid-associated disorders such as dyslipidemia and atherosclerosis and, more specifically, to compositions and methods for the treatment of lipid-associated disorders with a reduced flushing side effect.

### BACKGROUND OF THE INVENTION

Atherosclerosis is a process where deposits of fatty substances, cholesterol and other substances build up in the inner lining of an artery. This buildup is called plaque. Plaques that rupture cause blood clots to form that can block blood flow to the heart (heart attack) or the brain (stroke). Heart attack is the number one cause of death for both men and women in the United States and stroke is the number three cause of death [see, for example, Nature Medicine, Special Focus on Atherosclerosis, (2002) 8:1209-1262]. Abnormally high levels of circulating lipids are a major predisposing factor in development of atherosclerosis. Elevated levels of low density lipoprotein (LDL) cholesterol, elevated levels of triglycerides, or low levels of high density lipoprotein (HDL) cholesterol are, independently, risk factors for atherosclerosis and associated pathologies.

Niacin (nicotinic acid, pyridine-3-carboxylic acid, vitamin B3) is a water-soluble vitamin required by the human body for health, growth and reproduction. Niacin is also one of the oldest used drugs for the treatment of lipid-associated disorders. It is a valuable drug in that it favorably affects virtually all of the lipid parameters listed above [Goodman and Gilman's Pharmacological Basis of Therapeutics, editors Harmon J G and Limbird L E, Chapter 36, Mahley R W and Bersot T P (2001) pages 971-1002]. The benefits of niacin in the treatment or prevention of atherosclerotic cardiovascular disease have been documented in six major clinical trials [Guyton J R (1998) Am J Cardiol 82:18U-23U]. Structure and synthesis of analogs or derivatives of niacin are discussed throughout the Merck Index, An Encyclopedia of Chemicals, Drugs, and Biologicals, Tenth Edition (1983).

Unfortunately, the doses of niacin required to alter serum lipid levels can be quite large and at these dosages adverse side effects are frequent. Side effects can include gastrointestinal disturbances, liver toxicity, and disruption of glucose metabolism and uric acid levels. However, the most frequent and prominent side effect of niacin therapy is intense flushing, often accompanied by cutaneous itching, tingling and warmth. Although the flushing reaction is generally harmless, it is sufficiently unpleasant that patient compliance is markedly reduced. Often, 30-40% of patients cease taking niacin treatment within days after initiating therapy.

Efforts have been undertaken to develop niacin analogs, dosage forms and treatment protocols which minimize the cutaneous flush reaction while maintaining therapeutic efficacy. However, to date, these efforts have resulted in compounds or methods that only partially reduce the cutaneous flush reaction. In addition, these compounds or methods can result in other side effects. For example, compounds such as aspirin can be administered before administering niacin in an attempt to reduce flushing. However, at best, aspirin only results in a partial reduction of flushing in some patients, and the gastrointestinal side effects of aspirin limit its use. In addition, extended or sustained release formulations of niacin have been developed that reportedly have a lower incidence of flushing. However, these extended or sustained release formulations have been shown to result in liver toxicity which is a more severe side effect than flushing.

Thus, there exists a need for compounds and methods of identifying compounds that are anti-lypolytic without causing the level of flushing seen with niacin treatment. The present invention satisfies this need and provides related advantages as well.

### SUMMARY OF THE INVENTION

Applicants disclose herein that the ability of a niacin receptor modulator to activate the MAP kinase pathway in a cell is predictive of whether the modulator will cause flushing in vivo. Specifically, Applicants disclose that niacin receptor modulators that activate mitogen-activated protein kinase (MAP kinase) to a lesser extent than niacin cause less flushing than niacin in vivo. The in vivo flushing assay is labor intensive, time consuming and expensive, thus the ability to screen modulators for their flushing ability using an efficient in vitro cell based assay, such as the MAP kinase assay, is highly advantageous.

In a first aspect, the invention provides a method of identifying a niacin receptor modulator with reduced flushing effect compared to niacin or a niacin analog, comprising determining the MAP kinase activity of said modulator, wherein a decrease in MAP kinase activity induced by said modulator compared to MAP kinase activity induced by niacin or a niacin analog indicates that said modulator has reduced flushing effect when compared to niacin or a niacin analog. In one embodiment, said niacin receptor modulator is a niacin receptor agonist or partial agonist. In another embodiment, said niacin receptor modulator is an anti-lipolytic compound. In a further embodiment, said niacin receptor modulator has no significant flushing effect when compared to niacin or a niacin analog. In one embodiment, said decrease in MAP kinase activity induced by said modulator is at least two standard deviations below the level of MAP kinase activity induced by niacin or a niacin analog. In another embodiment, an antibody based assay is used to determine said MAP kinase activity. In a further embodiment, an enzyme-linked immunosorbent assay (ELISA) is used to determine said MAP kinase activity.

In a second aspect, the invention provides a method of identifying a niacin receptor modulator with reduced flushing effect compared to niacin, comprising determining the MAP kinase activity of said modulator, wherein a decrease in MAP kinase activity induced by said modulator compared to MAP kinase activity induced by niacin indicates that said modulator has reduced flushing effect when compared to niacin. In one embodiment, said niacin receptor modulator is a niacin receptor agonist or partial agonist. In another embodiment, said niacin receptor modulator is an anti-lipolytic compound. In a further embodiment, said niacin receptor modulator has no significant flushing effect when compared to niacin. In one embodiment, said decrease in MAP kinase activity induced by said modulator is at least two standard deviations below the level of MAP kinase activity induced by niacin. In another embodiment, an antibody based assay is used to determine said MAP kinase activity. In a further embodiment, an ELISA is used to determine said MAP kinase activity.

In a third aspect, the invention provides a method of identifying a niacin receptor modulator with reduced flushing effect compared to niacin, comprising: a) identifying a niacin receptor modulator, and b) determining the MAP kinase activity of said modulator, wherein a decrease in MAP kinase activity induced by said modulator compared to MAP kinase activity induced by niacin indicates that said modulator has reduced flushing effect when compared to niacin. In one embodiment, said niacin receptor modulator is a niacin receptor agonist or partial agonist. In another embodiment, said niacin receptor modulator is an anti-lipolytic compound. In a further embodiment, said niacin receptor modulator has no significant flushing effect when compared to niacin. In one embodiment, said decrease in MAP kinase activity induced by said modulator is at least two standard deviations below the level of MAP kinase activity induced by niacin. In another embodiment, an antibody based assay is used to determine said MAP kinase activity. In a further embodiment, an ELISA is used to determine said MAP kinase activity.

In a fourth aspect, the invention provides a niacin receptor modulator with reduced flushing effect compared to niacin or a niacin analog, wherein said modulator is identified as a modulator with reduced flushing effect compared to niacin or a niacin analog according to the method of the first aspect. In one embodiment, said modulator is a niacin receptor agonist or partial agonist. In another embodiment, said modulator is an anti-lipolytic compound. In a further embodiment, said modulator has no significant flushing effect when compared to niacin or a niacin analog.

In a fifth aspect, the invention provides a method for preparing a composition which comprises identifying a niacin receptor modulator with reduced flushing effect compared to niacin or a niacin analog and then admixing said modulator with a carrier, wherein said modulator is identified by the method of the first aspect.

In a sixth aspect, the invention provides a pharmaceutical composition comprising, consisting essentially of, or consisting of the modulator of the fourth aspect.

In a seventh aspect, the invention provides a method for preventing or treating a lipid-associated disorder in a subject, comprising administering to said subject an effective lipid altering amount of a niacin receptor modulator with reduced flushing effect compared to niacin or a niacin analog where said modulator is identified by the method of the first aspect. In one embodiment, said lipid-associated disorder is dyslipidemia, hyperlipidemia, hypertriglyceridemia, hypercholesterolemia, atherosclerosis, metabolic syndrome, heart disease, stroke, or peripheral vascular disease. In another embodiment, said lipid-associated disorder is dyslipidemia. In a further embodiment, said lipid-associated disorder is atherosclerosis. In one embodiment, the method of the seventh aspect further comprises administering to said subject an effective amount of an agent used for the treatment of obesity or diabetes in combination with an effective amount of niacin receptor modulator with reduced flushing effect compared to niacin or a niacin analog where said modulator is identified by the method of the first aspect. In one embodiment, the subject is a mammal and in another embodiment the subject is a human.

In an eighth aspect, the invention provides a method for decreasing LDL levels in a subject in need thereof, comprising administering to said subject an effective amount of the modulator of the fourth aspect.

In a ninth aspect, the invention provides a method for decreasing triglyceride levels in a subject in need thereof, comprising administering to said subject an effective amount of the modulator of the fourth aspect.

In a tenth aspect, the invention provides a method for increasing HDL levels in a subject in need thereof, comprising administering to said subject an effective amount of the modulator of the fourth aspect.

In an eleventh aspect, the invention provides a method for the manufacture of a medicament comprising the modulator of the fourth aspect, for use as a lipid altering agent. In addition, in the eleventh aspect the invention provides a method for the manufacture of a medicament comprising the modulator of the fourth aspect, for use in the treatment of a lipid-associated disorder.

### BRIEF DESCRIPTION OF THE DRAWINGS

FIG. 1 shows niacin-induced MAP kinase activation in CHO cells expressing the human niacin receptor by ELISA and Western Blot.

FIG. 2 shows MAP kinase activation in CHO cells expressing the human niacin receptor by niacin and Compound 8 using an ELISA.

FIG. 3 shows a table comparing niacin (Compound 1) and several niacin receptor modulators using the following assays: in vivo flushing in mice, cAMP assay in Chinese Hamster Ovary (CHO) cells expressing the human niacin receptor, and MAP kinase activation in CHO cells expressing the human niacin receptor by ELISA. The last column shows the ratio of the MAP kinase EC₅₀ to cAMP IC₅₀.

FIG. 4 shows a graph of the MAP kinase activation of several niacin receptor modulators in CHO cells expressing the human niacin receptor. Large arrows indicate compounds that have been shown to flush in mice and small arrows indicate compounds that do not flush in mice. The horizontal dotted line indicates 2 standard deviations below niacin.

FIG. 5 shows a niacin receptor modulator, Compound 11 that was selected for its low activation of MAP kinase on the human (top left panel) and mouse (top right panel) niacin receptor expressed in CHO cells. The compound was later shown to have no significant flushing in mice when compared to niacin (bottom right panel) and to decrease free fatty acid levels in mice (bottom left panel).

In an eighth aspect, the invention provides a method for decreasing LDL levels in a subject in need thereof, comprising administering to said subject an effective amount of the modulator of the fourth aspect.

In a ninth aspect, the invention provides a method for decreasing triglyceride levels in a subject in need thereof, comprising administering to said subject an effective amount of the modulator of the fourth aspect.

In a tenth aspect, the invention provides a method for increasing HDL levels in a subject in need thereof, comprising administering to said subject an effective amount of the modulator of the fourth aspect.

In an eleventh aspect, the invention provides a method for the manufacture of a medicament comprising the modulator of the fourth aspect, for use as a lipid altering agent.

In addition, in the eleventh aspect the invention provides a method for the manufacture of a medicament comprising the modulator of the fourth aspect, for use in the treatment of a lipid-associated disorder.

### BRIEF DESCRIPTION OF THE DRAWINGS

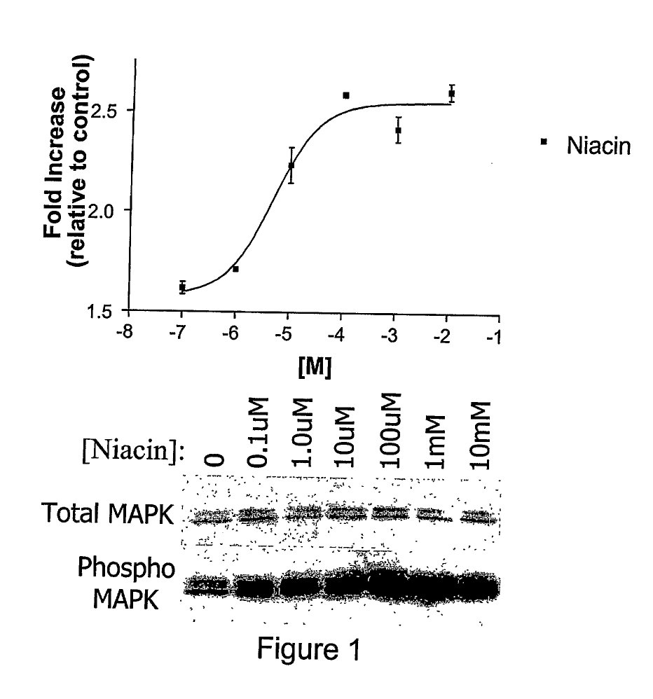

FIG. 1 shows niacin-induced MAP kinase activation in CHO cells expressing the human niacin receptor by ELISA and Western Blot.

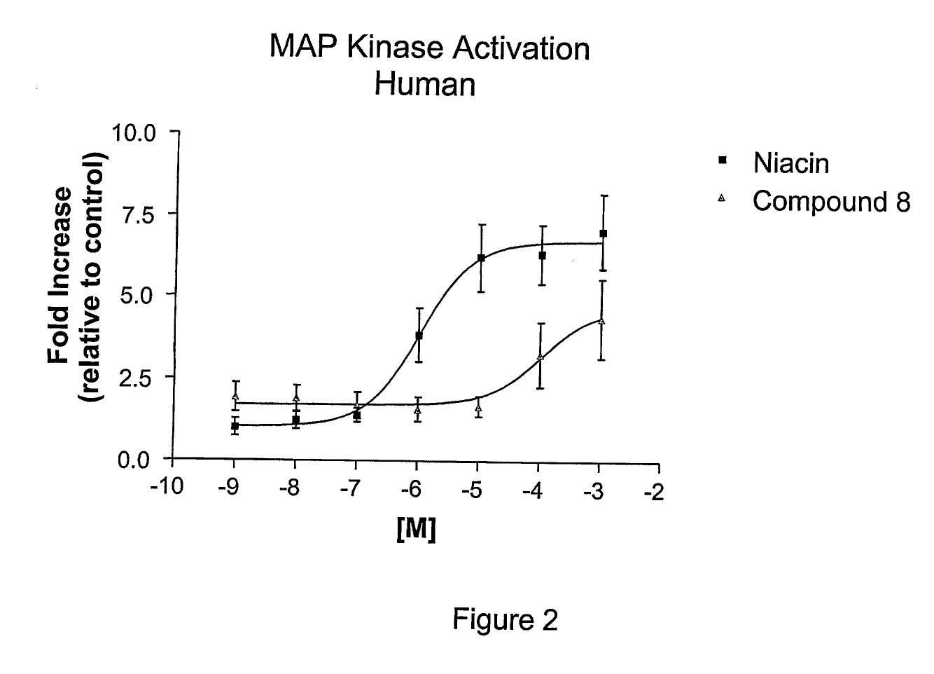

FIG. 2 shows MAP kinase activation in CHO cells expressing the human niacin receptor by niacin and Compound 8 using an ELISA.

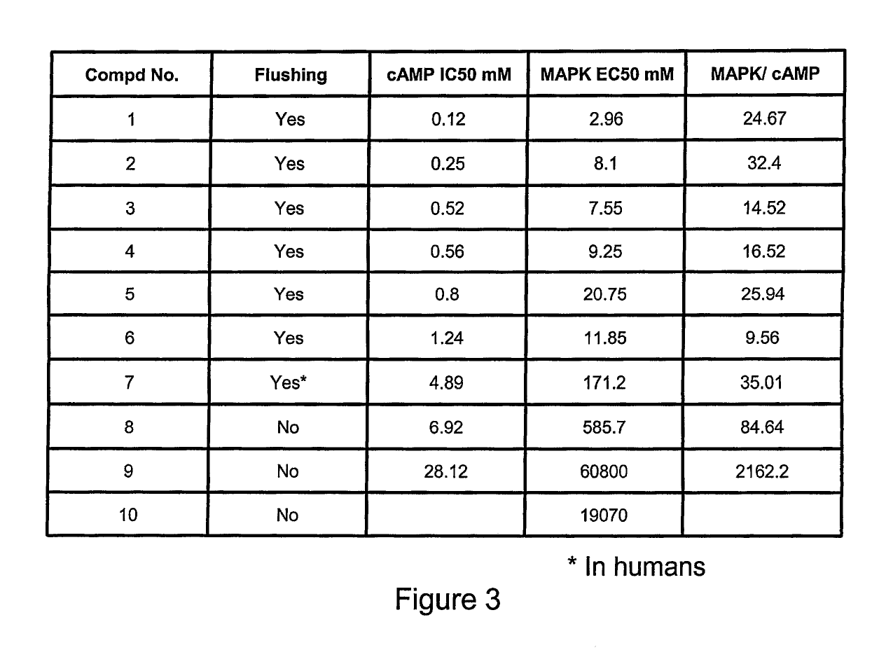

FIG. 3 shows a table comparing niacin (Compound 1) and several niacin receptor modulators using the following assays: in vivo flushing in mice, cAMP assay in Chinese Hamster Ovary (CHO) cells expressing the human niacin receptor, and MAP kinase activation in CHO cells expressing the human niacin receptor by ELISA. The last column shows the ratio of the MAP kinase EC₅₀ to cAMP IC₅₀.

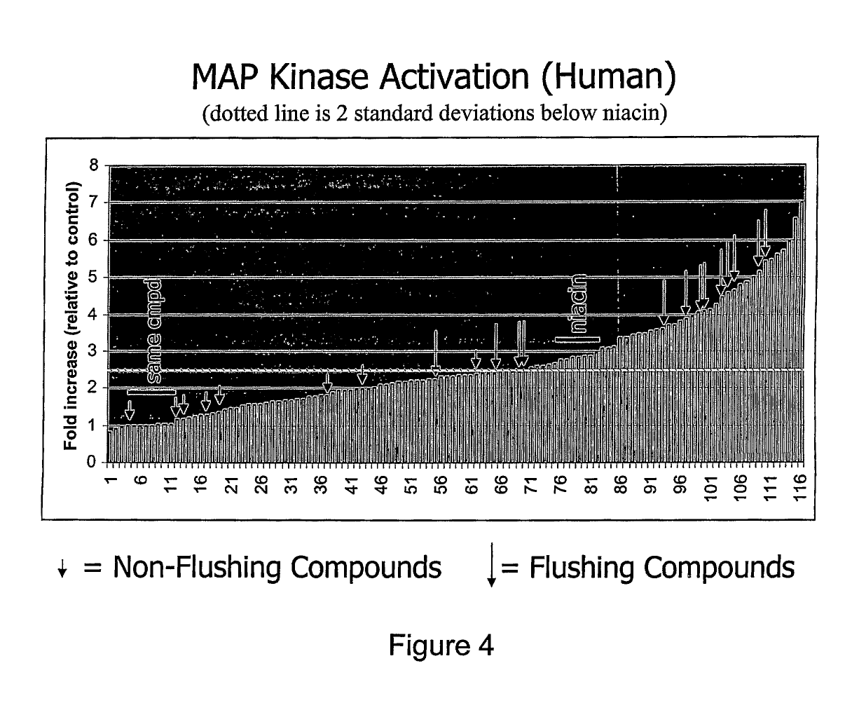

FIG. 4 shows a graph of the MAP kinase activation of several niacin receptor modulators in CHO cells expressing the human niacin receptor. Dark arrows indicate compounds that have been shown to flush in mice and light arrows indicate compounds that do not flush in mice. The horizontal dotted line indicates 2 standard deviations below niacin.

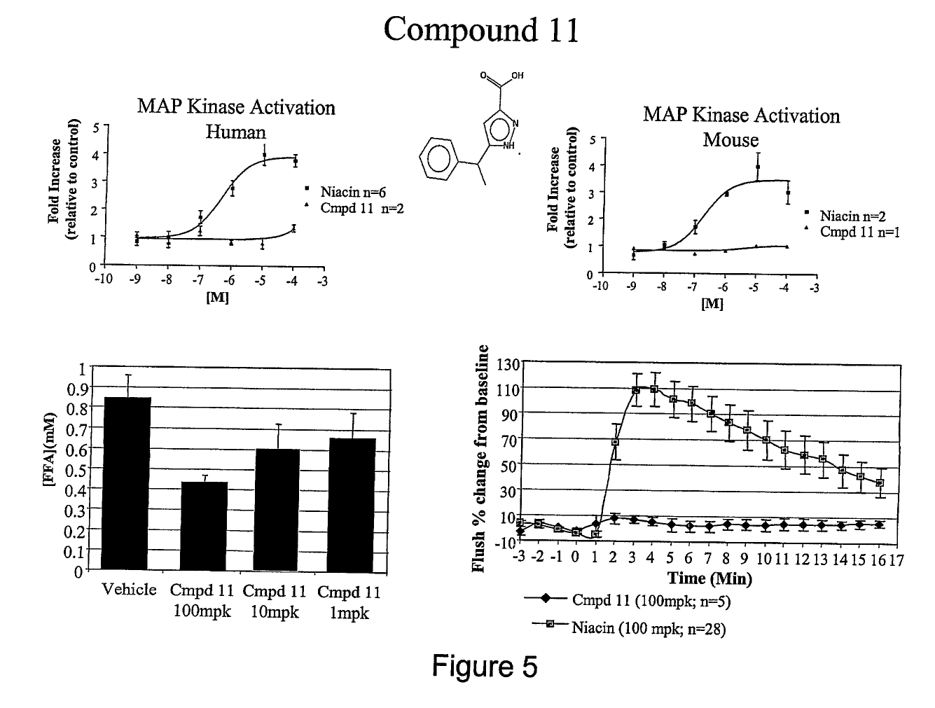

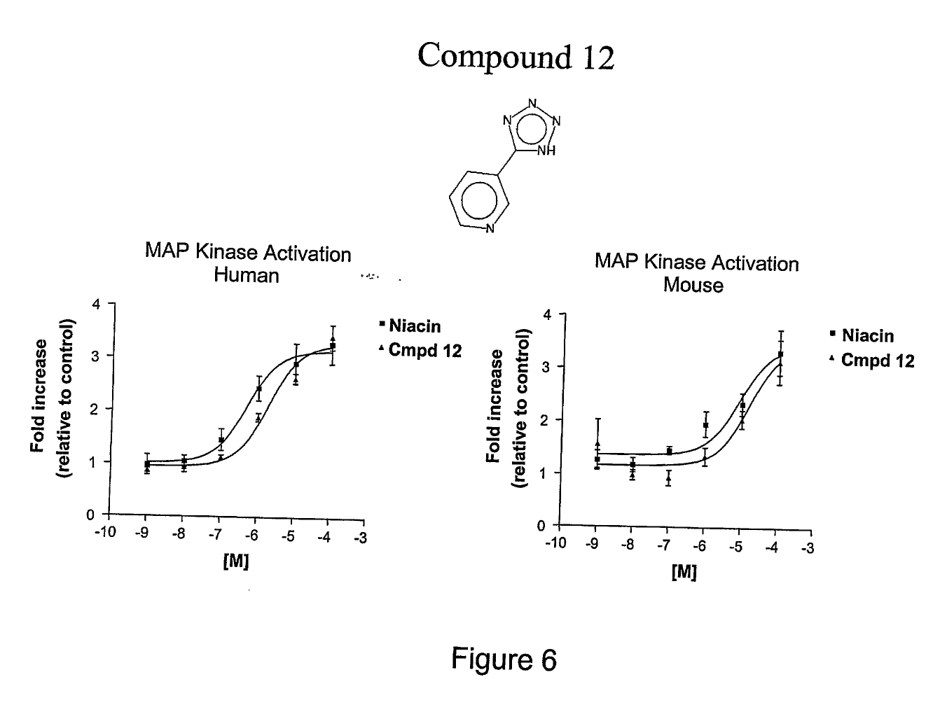

FIG. 5 shows a niacin receptor modulator, Compound 11 that was selected for its low activation of MAP kinase on the human (top left panel) and mouse (top right panel) niacin receptor expressed in CHO cells. The compound was later shown to have no significant flushing in mice when compared to niacin (bottom right panel) and to decrease free fatty acid levels in mice (bottom left panel).

FIG. 6 shows that a niacin analog compound, Compound 12, behaves like niacin in a MAP kinase assay on the human (left panel) and mouse (right panel) niacin receptor expressed in CHO cells.

### DETAILED DESCRIPTION

Applicants have discovered that the ability of a niacin receptor modulator to activate the MAP kinase pathway in cells correlates to the ability of the modulator to induce flushing in vivo. As shown herein, niacin binds to the human niacin receptor and activates MAP kinase within these cells (see Example 1 and FIG. 1). MAP kinase activation was shown using two types of antibody based assays, an ELISA and a Western Blot assay. As disclosed herein, niacin receptor modulators which are known to cause flushing in vivo were shown to have higher levels of MAP kinase activation compared to modulators which did not cause flushing in vivo (see Example 2 and FIGS. 3 and 4). Applicants then chose niacin receptor modulators with unknown ability to cause flushing in vivo and tested these modulators for their ability to activate MAP kinase in cells expressing the human or mouse niacin receptor (see Example 3). FIG. 5 shows a representative modulator, Compound 11, which had been selected based on the cAMP assay. This modulator was then tested in the MAP kinase ELISA assay and showed a low level of MAP kinase activation compared to niacin (see upper panels). Compound 11 was then tested for its ability to cause flushing in vivo. As shown in FIG. 5, lower right panel, Compound 11 did not cause any significant flushing in vivo in mice. In addition, as disclosed herein, Compound 11 is anti-lipolytic (see lower left panel). Applicants also disclose herein that niacin analogs which behave like niacin in a MAP kinase assay can be used in lieu of niacin in the methods of the invention. Such a compound, Compound 12, is shown in FIG. 6.

Although niacin has been used as a therapy for lipid-associated disorders for several years, the receptor through which niacin acted was not known until recently. Initially, it was suggested that niacin may act through a specific GPCR (Lorenzen A, et al. (2001) Molecular Pharmacology 59:349-357). Eventually, a known orphan GPCR called HM74a was identified as the nicotinic acid receptor (see, for example, U.S. application Ser. No. 10/314,048). The nucleotide sequence of the human niacin receptor can be found at GenBank Accession No. NM_{—}177551 and herein as SEQ ID NO:1.

Generally, when a ligand binds with its receptor, often referred to as activation of the receptor, there is a change in the conformation of the receptor that facilitates coupling between the intracellular region and an intracellular G-protein. Although other G proteins exist, currently, Gq, Gs, Gi, Gz and Go are G proteins that have been identified. There are also promiscuous G proteins, which appear to couple several classes of GPCRs to the phospholipase C pathway, such as Gα15 or Gα16 [Offermanns & Simon, J Biol Chem (1995) 270:15175-80], or chimeric G proteins designed to couple a large number of different GPCRs to the same pathway [Milligan & Rees, Trends in Pharmaceutical Sciences (1999) 20:118-24]. Ligand-activated GPCR coupling with the G-protein initiates a signaling cascade process referred to as signal transduction. Under normal conditions, signal transduction ultimately results in cellular activation or cellular inhibition.

The niacin receptor is known to couple to the Gi G protein, thus agonism of the niacin receptor results in a decrease in the level of intracellular cAMP. In adipose cells, a decrease in cAMP leads to a decrease in hormone sensitive lipase activity and a decrease in free fatty acid release. The consequence of decreasing free fatty acids is two-fold. First, it will ultimately lower LDL-cholesterol and raise HDL-cholesterol levels, which are independent risk factors for atherosclerosis and related disorders. Second, it will provide an increase in insulin sensitivity in individuals with insulin resistance or type 2 diabetes. Unfortunately, the use of niacin as a therapeutic is partially limited by a number of associated adverse side effects such as flushing.

Applicants disclose herein a method of identifying a compound with reduced flushing effect compared to niacin or a niacin analog by determining the MAP kinase activity of the compound, where a decrease in MAP kinase activity induced by the compound compared to MAP kinase activity induced by niacin or a niacin analog indicates that the compound has reduced flushing effect when compared to niacin or a niacin analog. For example, Applicants disclose a method of predicting whether a compound will have a reduced flushing effect compared to niacin or a niacin analog by determining the MAP kinase activity of the compound, where a decrease in MAP kinase activity induced by the compound compared to MAP kinase activity induced by niacin or a niacin analog indicates that the compound has reduced flushing effect when compared to niacin or a niacin analog.

The invention provides a method of identifying a niacin receptor modulator with reduced flushing effect compared to niacin or a niacin analog, comprising determining the MAP kinase activity of said modulator, wherein a decrease in MAP kinase activity induced by said modulator compared to MAP kinase activity induced by niacin or a niacin analog indicates that said modulator has reduced flushing effect when compared to niacin or a niacin analog. In one embodiment, said niacin receptor modulator is a niacin receptor agonist or partial agonist. In another embodiment, said niacin receptor modulator is an anti-lipolytic compound. In a further embodiment, said niacin receptor modulator has no significant flushing effect when compared to niacin or a niacin analog. In one embodiment, said decrease in MAP kinase activity induced by said modulator is at least two standard deviations below the level of MAP kinase activity induced by niacin or a niacin analog. In another embodiment, an antibody based assay is used to determine said MAP kinase activity. In a further embodiment, an ELISA is used to determine said MAP kinase activity.

The invention also provides a method of identifying a niacin receptor modulator with reduced flushing effect compared to niacin, comprising determining the MAP kinase activity of said modulator, wherein a decrease in MAP kinase activity induced by said modulator compared to MAP kinase activity induced by niacin indicates that said modulator has reduced flushing effect when compared to niacin. In one embodiment, said niacin receptor modulator is a niacin receptor agonist or partial agonist. In another embodiment, said niacin receptor modulator is an anti-lipolytic compound. In a further embodiment, said niacin receptor modulator has no significant flushing effect when compared to niacin. In one embodiment, said decrease in MAP kinase activity induced by said modulator is at least two standard deviations below the level of MAP kinase activity induced by niacin. In another embodiment, an antibody based assay is used to determine said MAP kinase activity. In a further embodiment, an ELISA is used to determine said MAP kinase activity.

Applicants disclose herein a method of identifying a compound with reduced flushing effect compared to niacin or a niacin analog by a) identifying a niacin receptor modulator, and b) determining the MAP kinase activity of the compound, where a decrease in MAP kinase activity induced by the compound compared to MAP kinase activity induced by niacin indicates that the compound has reduced flushing effect when compared to niacin. For example, Applicants disclose a method of predicting whether a compound will have a reduced flushing effect compared to niacin or a niacin analog by a) identifying a niacin receptor modulator, and b) determining the MAP kinase activity of the compound, where a decrease in MAP kinase activity induced by the compound compared to MAP kinase activity induced by niacin indicates that the compound has reduced flushing effect when compared to niacin. Applicants disclose a method of identifying a niacin receptor modulator with reduced flushing effect compared to niacin or a niacin analog, by: a) identifying a niacin receptor modulator, and b) determining the MAP kinase activity of said modulator, wherein a decrease in MAP kinase activity induced by said modulator compared to MAP kinase activity induced by niacin or a niacin analog indicates that said modulator has reduced flushing effect when compared to niacin or a niacin analog.

The invention further provides a method of identifying a niacin receptor modulator with reduced flushing effect compared to niacin, comprising: a) identifying a niacin receptor modulator, and b) determining the MAP kinase activity of said modulator, wherein a decrease in MAP kinase activity induced by said modulator compared to MAP kinase activity induced by niacin indicates that said modulator has reduced flushing effect when compared to niacin. In one embodiment, said niacin receptor modulator is a niacin receptor agonist or partial agonist. In another embodiment, said niacin receptor modulator is an anti-lipolytic compound. In a further embodiment, said niacin receptor modulator has no significant flushing effect when compared to niacin. In one embodiment, said decrease in MAP kinase activity induced by said modulator is at least two standard deviations below the level of MAP kinase activity induced by niacin. In another embodiment, an antibody based assay is used to determine said MAP kinase activity. In a further embodiment, an ELISA is used to determine said MAP kinase activity.

As used herein, the term “flushing” means a detectable cutaneous vasodilation reaction. For example, flushing can be caused by administration of a niacin receptor agonist such as niacin or a niacin analog. Niacin-induced flushing is thought to be mediated through prostaglandins such as prostaglandin D2 (PGD₂). A flushing reaction is characterized by redness of the skin and can also include other symptoms, for example, cutaneous itching, tingling, a feeling of warmth, or headache. The flushing reaction can occur anywhere on the skin, for example, on the face, neck or trunk, and can occur in one location or at more than one location. In humans, the flushing reaction can last from several minutes to a several hours. Generally, in humans a flushing reaction caused by oral administration of sufficient doses of niacin or a niacin analog can last anywhere from 20 minutes to 8 hours or more. In a mouse or rat, the flushing reaction usually peaks at about 3 minutes post administration of niacin (by injection) and declines significantly after about 30 minutes.

The amount of niacin or a niacin analog required to produce a detectable flushing reaction depends on several variables, for example, the formulation of the compound and the individual subject. In particular, the amount of niacin or a niacin analog required to produce a detectable flushing reaction can be dependent on, for example, the body weight of the individual, genetic makeup of the individual or general health of the individual. Amounts of niacin or a niacin analog that can cause a flushing reaction in a human can be less than those required to lower the amount of atherosclerosis associated serum lipids and can include, for example, at least 175 mg per day, at least 200 mg per day, at least 250 mg per day, at least 500 mg per day, at least 750 mg per day, at least 1 g per day, at least 1.5 g per day, at least 2 g per day, at least 2.5 g per day, at least 3 g per day, at least 3.5 g per day, at least 4 g per day, at least 4.5 g per day, at least 5 g per day, at least 5.5 g per day, at least 6 g per day, at least 6.5 g per day, at least 7 g per day, at least 7.5 g per day, at least 8 g per day, or more. For example, 500 mg to 2 g or more per day of niacin can cause a flushing reaction in most humans.

As used herein, “niacin” means nicotinic acid which has the following chemical formula:

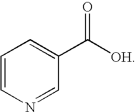

The term niacin also includes pharmaceutically acceptable salts and solvates of niacin which have similar properties to the free acid form of niacin. As understood by one skilled in the art, niacin can be formulated with other compounds such that its pharmacologic properties are modified. For example, niacin can be formulated as an immediate release (IR) form or as an extended or sustained release (SR) form depending on other compounds that are added to the niacin.

Extended or sustained release formulations are designed to slowly release the active ingredient from the tablet or capsule, which allows a reduction in dosing frequency as compared to the typical dosing frequency associated with conventional or immediate dosage forms. The slow drug release is designed to reduce and prolong blood levels of the drug and, thus, minimize or lessen the flushing side effects that are associated with conventional or immediate release niacin products. However, studies in patients with lipid-associated disorders have demonstrated that some extended or sustained release products do not have the same advantageous lipid-altering effects as immediate release niacin, and in fact have a worse side effect profile compared to the immediate release product. For example, extended or sustained release niacin formulations are known to cause greater incidences of liver toxicity, as described in Henken et al.: Am J Med, 91:1991 (1991) and Dalton et al.: Am J Med, 93:102 (1992). Extended or sustained release formulations of niacin have been developed, such as Nicobid® capsules (Rhone-Poulenc Rorer), Endur-acin® (Innovite Corporation), and the formulations described in U.S. Pat. Nos. 5,126,145 and 5,268,181, which describe sustained release niacin formulations containing two different types of hydroxy propyl methylcelluloses and a hydrophobic component.

As used herein, “niacin analog” means a compound structurally or functionally related to niacin which has a similar MAP kinase profile and flushing effect as niacin. An example of a niacin analog is Compound 12 (see FIG. 6). This compound is structurally related to niacin and differs from niacin by containing a tetrazole group. In addition, this compound is functionally related to niacin since it binds to the niacin receptor. Further, this compound has a similar MAP kinase profile (see FIG. 6) and flushing effect as niacin. Therefore, this compound can be used in the methods of the invention as a reference instead of niacin. Other such niacin analogs will be apparent to those of skill in the art.

Several structural analogs of niacin are known in the art. In some embodiments, structural analogues of niacin contain at least one functional acidic group, such as carboxyl, tetrazolyl, and the like. In some embodiments, structural analogues of niacin contain at least one nitrogen ring atom, such as the nitrogen present in pyridinyl, pyrazolyl, isoxazolyl, and the like. In some embodiments, structural analogues of niacin contain at least one functional acidic group and at least one nitrogen ring atom. These groups include pro-drug groups that are transformed in vivo to yield the functional acidic group or ring nitrogen, for example, by hydrolysis in blood. A thorough discussion is provided in T. Higuchi and V. Stella, “Pro-drugs as Novel Delivery Systems,” Vol. 14 of the A.C.S. Symposium Series, and in “Bioreversible Carriers in Drug Design,” ed. Edward B. Roche, American Pharmaceutical Association and Pergamon Press, 1987, both of which are hereby incorporated by reference.

A niacin analog can be functionally related to niacin, for example, a niacin analog can have a function of niacin such as specifically binding to the niacin receptor or initiating an intracellular signal in response to binding at the niacin receptor. For example, a niacin analog can be a niacin receptor agonist.

Several analogs or derivatives of niacin are known in the art and can be found, for example, in Merck Index, An Encyclopedia of Chemicals, Drugs, and Biologicals, Tenth Edition (1983). As described above for niacin, niacin analogs can be formulated in different ways to modify their pharmacologic properties.

A “niacin receptor modulator” is material, for example, a ligand or compound, which modulates or changes an intracellular response when it binds to a niacin receptor. An intracellular response can be, for example, a change in GTP binding to membranes or modulation of the level of a second messenger such as cAMP.

An “agonist” is material, for example, a ligand or compound, which activates an intracellular response when it binds to the receptor. An intracellular response can be, for example, enhancement of GTP binding to membranes or modulation of the level of a second messenger such as cAMP. In some embodiments, an agonist is material not previously known to activate the intracellular response when it binds to the receptor (for example, to enhance GTPγS binding to membranes or to lower intracellular cAMP level).

A “partial agonist” is material, for example, a ligand or compound, which activates an intracellular response when it binds to the receptor but to a lesser degree or extent than a full agonist.

As used herein, a “niacin receptor partial agonist” is material that activates an intracellular response when it binds to a niacin receptor, but to a lesser degree than niacin which is a full agonist at the niacin receptor. Technically, the term partial agonist is a relative term because a partial agonist generates a partial response compared to a full agonist. Since new compounds are being discovered with time, the full agonist can change and a formerly full agonist can become a partial agonist. For clarity, a niacin receptor partial agonist as used herein is compared to niacin as the full agonist. A niacin receptor partial agonist has a detectably lesser degree of activation of an intracellular response compared to the niacin, i.e. a niacin receptor partial agonist elicits less than a maximal response. Thus, a niacin receptor partial agonist has less efficacy than niacin. For example, a niacin receptor partial agonist has 90% or less efficacy compared to niacin, 85% or less efficacy compared to niacin, 80% or less efficacy compared to niacin, 75% or less efficacy compared to niacin, 70% or less efficacy compared to niacin, 65% or less efficacy compared to niacin, 60% or less efficacy compared to niacin, 55% or less efficacy compared to niacin, 50% or less efficacy compared to niacin, 45% or less efficacy compared to niacin, 40% or less efficacy compared to niacin, 35% or less efficacy compared to niacin, 30% or less efficacy compared to niacin, 25% or less efficacy compared to niacin, 20% or less efficacy compared to niacin, 15% or less efficacy compared to niacin, or 10% efficacy compared to niacin. For example, a niacin receptor partial agonist can have 10% to 90% efficacy compared to niacin, 20% to 80% efficacy compared to niacin, 30% to 70% efficacy compared to niacin, 40% to 60% efficacy compared to niacin, or 45% to 55% efficacy compared to niacin. Efficacy, which is the magnitude of the measured response, is different from potency which is the amount of compound it takes to elicit a defined response. Therefore, a niacin receptor partial agonist can be more, less, or equally potent when compared to an agonist, antagonist, or inverse agonist.

A niacin receptor partial agonist can be determined using assays well known in the art. For example, a niacin receptor partial agonist can be determined using a cAMP assay.

Regarding the niacin receptor, several niacin receptor sequences are known in the art. For example, a human niacin receptor nucleotide sequence can be found at GenBank Accession No. NM_{—}177551 and is listed herein as SEQ ID NO:1. It is also understood that limited modifications to the niacin receptor can be made without destroying the ability of a niacin receptor to bind niacin. For example, niacin receptor is intended to include other niacin receptor polypeptides, for example, species homologues of the human niacin receptor polypeptide (SEQ ID NO: 2). The sequence of species homologs of the human niacin receptor are present in the database, for example, a rat homolog of the niacin receptor can be found in GenBank at Accession No. BAC58009. In addition, a niacin receptor includes splice variants and allelic variants of niacin receptors that retain substantially the niacin receptor function of the entire niacin receptor polypeptide.

Further, a niacin receptor can contain amino acid changes, for example, conservative amino acid changes, compared to the wild-type receptor so long as the mutated receptor retains substantially the niacin receptor function of the wild-type niacin receptor polypeptide. Conservative and non-conservative amino acid changes, gaps, and insertions to an amino acid sequence can be compared to a reference sequence using available algorithms and programs such as the Basic Local Alignment Search Tool (“BLAST”) using default settings (See, e.g., Karlin and Altschul, Proc Natl Acad Sci USA (1990) 87:2264-8; Altschul et al., J Mol Biol (1990) 215:403-410; Altschul et al., Nature Genetics (1993) 3:266-72; and Altschul et al., Nucleic Acids Res (1997) 25:3389-3402).

The niacin receptor specifically binds to niacin. The term specifically binds is intended to mean the polypeptide will have an affinity for a target polypeptide that is measurably higher than its affinity for an un-related polypeptide. Several methods for detecting or measuring receptor binding are well known in the art, for example, radio-ligand binding assays, or assays with a functional read-out such as a FLIPR assay.

It is understood that a fragment of a niacin receptor which retains substantially the niacin receptor function of the entire polypeptide can be used in lieu of the entire polypeptide. For example, a ligand binding domain of a niacin receptor can be used in lieu of the entire polypeptide in order to determine binding of a partial agonist to a niacin receptor.

“Reduced” means a decrease in a measurable quantity or a particular activity and is used synonymously with the terms “decreased”, “diminishing”, “lowering”, and “lessening.” In reference to an amount of flushing, a reduced flushing effect can be, for example, a decrease in the severity of flushing and/or fewer flushing events than would otherwise occur (a decrease in the incidence of flushing). For example, the severity and/or incidence of flushing can be decreased at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99%. In addition, flushing can be decreased 100% or eliminated such that no significant flushing is detectable. In one embodiment, the intensity of flushing is decreased at least about 80%. In another embodiment, the decrease in flushing is a complete reduction or elimination of flushing.

Several methods can be used to detect and quantify flushing. For example, flushing can be visually detected and quantified. One method for detecting and quantifying flushing is by Laser Doppler, for example using a Pirimed PimII Laser Dopler. In addition, surveys of subjects can be taken to assess flushing and the severity of symptoms that can be associated with flushing such as tingling or a feeling of warmth. Another method for detecting and quantifying flushing can include measurement of the level of prostaglandin D₂ (PGD₂) or prostaglandin F₂ (PGF₂) in a biological sample from a subject such as blood or urine. In addition, for example, the level of PGD-M, the major urinary metabolite of PGD₂ can be measured from the urine of subjects. Assays for measuring prostaglandin levels are commercially available, for example, an enzyme immunoassay for PGD₂ is available from Cayman Chemical (Ann Arbor, Mich.).

In one embodiment, in the methods of the invention, the decrease in MAP kinase activity induced by the compound or modulator is at least two standard deviations below the level of MAP kinase activity induced by niacin or a niacin analog. As shown in FIG. 4, a decrease in MAP kinase activity of at least two standard deviations below the level of MAP kinase activity induced by niacin identified all of the known non-flushing compounds indicated by the light arrows. As understood by one skilled in the art, different cut-off values can be chosen depending on the needs of the artisan. For example, if one wanted to be sure to capture every non-flushing compound, one may choose to err on the side of identifying more compounds with the idea that some may not end up a having reduced flushing effect when tested in vivo. In such a case one may set the cut-off above two standard deviations from niacin or a niacin analog, for example, 1.5 standard deviations below niacin or a niacin analog. Conversely, one may want to avoid any compound that has flushing activity and so the cut-off could be set below two standard deviations from niacin or a niacin analog, for example, 2.5 standard deviations below niacin or a niacin analog. In different embodiments of the invention, the decrease in MAP kinase activity induced by the modulator is at least 1, 1.2, 1.4, 1.6, 1.8, 1.9, 2.0, 2.1, 2.2, 2.3, 2.4, 2.5, 2.6, 2.7, 2.8, 2.9, 3.0, 3.2, 3.4, 3.6, 3.8, 4.0, 4.2, 4.4, 4.6, 4.8 or 5.0 standard deviations below the level of MAP kinase activity induced by niacin or a niacin analog. Cut-off values can also be expressed as ranges, for example, the decrease in MAP kinase activity induced by the modulator can be between, for example, 1 and at least 4, 2 and at least 3, or 2.5 and at least 3 standard deviations below the level of MAP kinase activity induced by niacin or a niacin analog.

Any assay to determine MAP kinase activity can be used in the methods of the invention. For example, a substrate activity assay such as an assay using mylein basic protein, which is a substrate for MAP kinase, can be used in the methods of the invention. In one embodiment, an antibody based assay can be used to determine MAP kinase activity. Such assays are well known in the art and include, for example, Western blot, ELISA, immunoprecipitation, fluorescent polarization assay (FPA), Biacore assay and the like. In one embodiment, the assay used to determine MAP kinase activity is an ELISA. In one embodiment, the assay used to determine MAP kinase activity in the methods of the invention uses the human niacin receptor.

The invention also provides a niacin receptor modulator with reduced flushing effect compared to niacin or a niacin analog, wherein said modulator is identified as a modulator with reduced flushing effect compared to niacin or a niacin analog according to the method of: determining the MAP kinase activity of said modulator, wherein a decrease in MAP kinase activity induced by said modulator compared to MAP kinase activity induced by niacin or a niacin analog indicates that said modulator has reduced flushing effect when compared to niacin or a niacin analog. For example, the invention provides a niacin receptor modulator with reduced flushing effect compared to niacin or a niacin analog, wherein said modulator is identified as a modulator with reduced flushing effect compared to niacin or a niacin analog according to the method of: determining the MAP kinase activity of said modulator, wherein a decrease in MAP kinase activity induced by said modulator compared to MAP kinase activity induced by niacin indicates that said modulator has reduced flushing effect when compared to niacin. In addition, the invention discloses a niacin receptor modulator with reduced flushing effect compared to niacin or a niacin analog, wherein said modulator is identified as a modulator with reduced flushing effect compared to niacin or a niacin analog according to the method of: a) identifying a niacin receptor modulator, and b) determining the MAP kinase activity of said modulator, wherein a decrease in MAP kinase activity induced by said modulator compared to MAP kinase activity induced by niacin indicates that said modulator has reduced flushing effect when compared to niacin.

In one embodiment, said modulator is a niacin receptor agonist or partial agonist. In another embodiment, said modulator is an anti-lipolytic compound. In a further embodiment, said modulator has no significant flushing effect when compared to niacin or a niacin analog.

The invention further provides a method for preparing a composition which comprises identifying a niacin receptor modulator with reduced flushing effect compared to niacin or a niacin analog and then admixing said modulator with a carrier, wherein said modulator is identified by determining the MAP kinase activity of said modulator, wherein a decrease in MAP kinase activity induced by said modulator compared to MAP kinase activity induced by niacin or a niacin analog indicates that said modulator has reduced flushing effect when compared to niacin or a niacin analog. In addition, the invention discloses a method for preparing a composition which comprises identifying a niacin receptor modulator with reduced flushing effect compared to niacin or a niacin analog and then admixing said modulator with a carrier, wherein said modulator is identified according to the method of: a) identifying a niacin receptor modulator, and b) determining the MAP kinase activity of said modulator, wherein a decrease in MAP kinase activity induced by said modulator compared to MAP kinase activity induced by niacin indicates that said modulator has reduced flushing effect when compared to niacin.

The invention also provides a pharmaceutical composition comprising, consisting essentially of, or consisting of a niacin receptor modulator with reduced flushing effect compared to niacin or a niacin analog, wherein said modulator is identified as a modulator with reduced flushing effect compared to niacin or a niacin analog according to the method of: determining the MAP kinase activity of said modulator, wherein a decrease in MAP kinase activity induced by said modulator compared to MAP kinase activity induced by niacin or a niacin analog indicates that said modulator has reduced flushing effect when compared to niacin or a niacin analog. The invention also discloses a pharmaceutical composition comprising, consisting essentially of, or consisting of a niacin receptor modulator with reduced flushing effect compared to niacin or a niacin analog, wherein said modulator is identified as a modulator with reduced flushing effect compared to niacin or a niacin analog according to the method of: a) identifying a niacin receptor modulator, and b) determining the MAP kinase activity of said modulator, wherein a decrease in MAP kinase activity induced by said modulator compared to MAP kinase activity induced by niacin indicates that said modulator has reduced flushing effect when compared to niacin.

As used herein “composition” means a material comprising at least one component. A pharmaceutical composition is an example of a composition. A pharmaceutical composition means a composition comprising at least one active ingredient, whereby the composition is amenable to investigation for a specified, efficacious outcome in a subject (for example, a human). Those of ordinary skill in the art will understand and appreciate the techniques appropriate for determining whether an active ingredient has a desired efficacious outcome based upon the needs of the artisan.

Compositions described herein can include a pharmaceutically or physiologically acceptable carrier. Suitable pharmaceutically-acceptable carriers are available to those in the art; for example, see Remington: The Science and Practice or Pharmacy, 20^{th} Edition, 2000, Lippincott, Williams & Wilkons, (Gennaro et al., eds.). While it is possible that, for use in prophylaxis or treatment, a compound of the invention can in an alternative use be administered as a raw or pure chemical, it can also be desirable to present the compound or active ingredient as a pharmaceutical formulation or composition.

The invention thus further provides pharmaceutical formulations comprising a compound of the invention or a pharmaceutically acceptable salt or derivative thereof together with one or more pharmaceutically acceptable carriers thereof and/or prophylactic ingredients. The carrier(s) are “acceptable” in the sense of being compatible with the other ingredients of the formulation and not overly deleterious to the recipient thereof.

Pharmaceutical formulations include those suitable for oral, rectal, nasal, topical (including buccal and sub-lingual), vaginal or parenteral (including intramuscular, sub-cutaneous and intravenous) administration or in a form suitable for administration by inhalation or insufflation.

The compounds of the invention, together with a conventional adjuvant, carrier, or diluent, can be placed into the form of pharmaceutical formulations and unit dosages thereof, and in such form can be employed as solids, such as tablets or filled capsules, or liquids such as solutions, suspensions, emulsions, elixirs, gels or capsules filled with the same, all for oral use, in the form of suppositories for rectal administration; in the form of liquids, gels, lotions or ointments for topical use, or in the form of sterile injectable solutions for parenteral (including subcutaneous) use. Such pharmaceutical compositions and unit dosage forms thereof can comprise conventional ingredients in conventional proportions, with or without additional active compounds or principles, and such unit dosage forms can contain any suitable effective amount of the active ingredient commensurate with the intended daily dosage range to be employed.

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substances which can also act as diluents, flavouring agents, solubilizers, lubricants, suspending agents, binders, preservatives, tablet disintegrating agents, or an encapsulating material. In powders, the carrier can be a finely divided solid which is in a mixture with the finely divided active component. In tablets, the active component can be mixed with the carrier having the necessary binding capacity in suitable proportions and compacted to the desire shape and size.

Powders and tablets can contain varying percentage amounts of the active compound. A representative amount in a powder or tablet can contain from 0.5 to about 90 percent of the active compound; however, an artisan would know when amounts outside of this range are necessary. Suitable carriers for powders and tablets are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges can be used as solid forms suitable for oral administration.

For preparing suppositories, a low melting wax, such as an admixture of fatty acid glycerides or cocoa butter, can be first melted and the active component can be dispersed homogeneously therein, as by stirring. The molten homogenous mixture can then poured into convenient sized molds, allowed to cool, and thereby to solidify. Formulations suitable for vaginal administration can be presented as pessaries, tampons, creams, gels, pastes, foams or sprays containing in addition to the active ingredient such carriers as are known in the art to be appropriate.

Liquid form preparations include solutions, suspensions, and emulsions, for example, water or water-propylene glycol solutions. For example, parenteral injection liquid preparations can be formulated as solutions in aqueous polyethylene glycol solution. Injectable preparations, for example, sterile injectable aqueous or oleaginous suspensions can be formulated according to the known art using suitable dispersing or wetting agents and suspending agents. The sterile injectable preparation can also be a sterile injectable solution or suspension in a nontoxic parenterally acceptable diluent or solvent, for example, as a solution in 1,3-butanediol. Among the acceptable vehicles and solvents that can be employed are water, Ringer's solution, and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid find use in the preparation of injectables.

The compositions according to the present invention can thus be formulated for parenteral administration (that is, by injection, for example, bolus injection or continuous infusion) and can be presented in unit dose form in ampoules, pre-filled syringes, small volume infusion or in multi-dose containers with an added preservative. The compositions can take such forms as suspensions, solutions, or emulsions in oily or aqueous vehicles, and can contain formulatory agents such as suspending, stabilizing and/or dispersing agents. Alternatively, the active ingredient can be in powder form, obtained by aseptic isolation of sterile solid or by lyophilization from solution, for constitution with a suitable vehicle, e.g. sterile, pyrogen-free water, before use.

Aqueous solutions suitable for oral use can be prepared by dissolving the active component in water and adding suitable colorants, flavors, stabilizing and thickening agents, as desired. Aqueous suspensions suitable for oral use can be made by dispersing the finely divided active component in water with viscous material, such as natural or synthetic gums, resins, methylcellulose, sodium carboxymethylcellulose, or other well known suspending agents. Also included are solid form preparations which are intended to be converted, shortly before use, to liquid form preparations for oral administration. Such liquid forms include solutions, suspensions, and emulsions. These preparations can contain, in addition to the active component, colorants, flavors, stabilizers, buffers, artificial and natural sweeteners, dispersants, thickeners, solubilizing agents, and the like.

For topical administration to the epidermis the compositions according to the invention can be formulated as ointments, creams or lotions, or as a transdermal patch. Ointments and creams can, for example, be formulated with an aqueous or oily base with the addition of suitable thickening and/or gelling agents. Lotions can be formulated with an aqueous or oily base and can, in general, also contain one or more emulsifying agents, stabilizing agents, dispersing agents, suspending agents, thickening agents, or coloring agents.

Formulations suitable for topical administration in the mouth include lozenges comprising active agent in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

Solutions or suspensions can be applied directly to the nasal cavity by conventional means, for example with a dropper, pipette or spray. The formulations can be provided in single or multi-dose form. In the latter case of a dropper or pipette, this can be achieved by the individual administering an appropriate, predetermined volume of the solution or suspension. In the case of a spray, this can be achieved for example by means of a metering atomizing spray pump. Administration to the respiratory tract can also be achieved by means of an aerosol formulation in which the active ingredient is provided in a pressurized pack with a suitable propellant. If a pharmaceutical composition is administered as an aerosol, for example a nasal aerosols or by inhalation, this can be carried out, for example, using a spray, a nebulizer, a pump nebulizer, an inhalation apparatus, a metered inhaler or a dry powder inhaler. Pharmaceutical forms for administration of the compositions of the invention as an aerosol can be prepared by processes well-known to the person skilled in the art. For their preparation, for example, solutions or dispersions of the compounds of the invention in water, water/alcohol mixtures or suitable saline solutions can be employed using customary additives, for example benzyl alcohol or other suitable preservatives, absorption enhancers for increasing the bioavailability, solubilizers, dispersants and others, and, if appropriate, customary propellants, for example include carbon dioxide, CFC's, such as, dichlorodifluoromethane, trichlorofluoromethane, or dichlorotetrafluoroethane; and the like. The aerosol can conveniently also contain a surfactant such as lecithin. The dose of drug can be controlled by provision of a metered valve.

In formulations intended for administration to the respiratory tract, including intranasal formulations, the compound will generally have a small particle size, for example, of the order of 10 microns or less. Such a particle size can be obtained by means known in the art, for example by micronization. When desired, formulations adapted to give sustained release of the active ingredient can be employed.

Alternatively the active ingredients can be provided in the form of a dry powder, for example, a powder mix of the compound in a suitable powder base such as lactose, starch, starch derivatives such as hydroxypropylmethyl cellulose and polyvinylpyrrolidone (PVP). Conveniently the powder carrier can form a gel in the nasal cavity. The powder composition can be presented in unit dose form, for example, in capsules or cartridges of, for example, gelatin, or blister packs from which the powder may be administered by means of an inhaler.

In addition to the formulations described previously, the compounds can also be formulated as a depot preparation. Such long acting formulations can be administered by implantation (for example, subcutaneously or intramuscularly) or by intramuscular injection. Thus, for example, the compounds can be formulated with suitable polymeric or hydrophobic materials (for example, as an emulsion in an acceptable oil) or ion exchange resins, or as sparingly soluble derivatives, for example, as a sparingly soluble salt. In addition, a composition can be delivered via a controlled release system such as a pump.

Additionally, the compositions can be delivered using a sustained-release system, such as semipermeable matrices of solid hydrophobic polymers containing the therapeutic agent. Various sustained release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for modulator stabilization can be employed.

Suitable routes of administration to a subject include oral, topical, nasal, rectal, transmucosal, or intestinal administration, parenteral delivery, including intra-muscular, subcutaneous, intramedullary injections, as well as intrathecal, direct intra-ventricular, intravenous, intraperitoneal, intranasal, intrapulmonary (inhaled) or intra-ocular injections using methods known in the art. Other routes of administration are aerosol and depot formulation. In one embodiment, route of administration is oral.

The invention provides a method for preventing or treating a lipid-associated disorder in a subject, comprising administering to said subject an effective lipid altering amount of a niacin receptor modulator with reduced flushing effect compared to niacin or a niacin analog identified by the method of: determining the MAP kinase activity of said modulator, wherein a decrease in MAP kinase activity induced by said modulator compared to MAP kinase activity induced by niacin or a niacin analog indicates that said modulator has reduced flushing effect when compared to niacin or a niacin analog. For example, the invention provides a method for preventing or treating a lipid-associated disorder in a subject, comprising administering to said subject an effective lipid altering amount of a niacin receptor modulator with reduced flushing effect compared to niacin identified by the method of: determining the MAP kinase activity of said modulator, wherein a decrease in MAP kinase activity induced by said modulator compared to MAP kinase activity induced by niacin indicates that said modulator has reduced flushing effect when compared to niacin. The invention also discloses a method for preventing or treating a lipid-associated disorder in a subject, comprising administering to said subject an effective lipid altering amount of a niacin receptor modulator with reduced flushing effect compared to niacin or a niacin analog identified by the method of: a) identifying a niacin receptor modulator, and b) determining the MAP kinase activity of said modulator, wherein a decrease in MAP kinase activity induced by said modulator compared to MAP kinase activity induced by niacin indicates that said modulator has reduced flushing effect when compared to niacin.

In one embodiment, said lipid-associated disorder is dyslipidemia, hyperlipidemia, hypertriglyceridemia, hypercholesterolemia, atherosclerosis, metabolic syndrome, heart disease, stroke, or peripheral vascular disease. In another embodiment, said lipid-associated disorder is dyslipidemia. In a further embodiment, said lipid-associated disorder is atherosclerosis.

As used herein the term “treating” in reference to a disorder means a reduction in severity of one or more symptoms associated with a particular disorder. Therefore, treating a disorder does not necessarily mean a reduction in severity of all symptoms associated with a disorder and does not necessarily mean a complete reduction in the severity of one or more symptoms associated with a disorder. Similarly, the term “preventing” means prevention of the occurrence or onset of one or more symptoms associated with a particular disorder and does not necessarily mean the complete prevention of a disorder. The methods of the invention can be used to treat a niacin-responsive disorder including, for example, a lipid-associated disorder as described herein.

As used herein a “subject” means any animal, including mammals, for example, mice, rats, other rodents, rabbits, dogs, cats, swine, cattle, sheep, horses, or primates, for example, humans. In one embodiment, a subject is a mammal. In another embodiment, a subject is a human.

As used herein the term “effective lipid altering amount” in reference to an amount of a niacin receptor modulator means an amount of modulator sufficient to detectably alter the amount of an atherosclerosis associated serum lipid, for example, a decrease in the amount of LDL-cholesterol, VLDL-cholesterol, serum lipoprotein (a) (Lp(a)), or triglycerides, or an increase in HDL-cholesterol in a subject. For example, an effective lipid altering amount of a niacin modulator can increase the amount of HDL-cholesterol or lower the amount of LDL-cholesterol. In addition, for example, an effective lipid altering amount of a niacin modulator can both increase the amount of HDL-cholesterol and lower the amount of LDL-cholesterol. Standard laboratory assays for measuring the amount of these lipids in the blood are well known in the art.

Cholesterol is transported in the blood by lipoprotein complexes, such as VLDL-cholesterol, LDL-cholesterol, and high density lipoprotein-cholesterol (HDL-cholesterol). LDL carries cholesterol in the blood to the subendothelial spaces of blood vessel walls. It is believed that peroxidation of LDL-cholesterol within the subendothelial space of blood vessel walls leads to atherosclerosis plaque formation. HDL-cholesterol, on the other hand, is believed to counter plaque formation and delay or prevent the onset of cardiovascular disease and atherosclerotic symptoms. Several subtypes of HDL-cholesterol, such as HDL₁-cholesterol, HDL₂-cholesterol and HDL₃-cholesterol, have been identified to date.

There are several mechanisms by which HDL may protect against the progression of atherosclerosis. Studies in vitro have shown that HDL is capable of removing cholesterol from cells [Picardo et al., (1986) Arteriosclerosis, 6, 434-441]. Data of this nature suggest that one antiatherogenic property of HDL may lie in its ability to deplete tissue of excess free cholesterol and eventually lead to the delivery of this cholesterol to the liver [Glomset, (1968) J. Lipid Res., 9, 155-167]. This has been supported by experiments showing efficient transfer of cholesterol from HDL to the liver [Glass et al., (1983) J. Biol. Chem., 258 7161-7167; McKinnon et al., (1986) J. Biol. Chem., 26, 2548-2552]. In addition, HDL may serve as a reservoir in the circulation for apoproteins necessary for the rapid metabolism of triglyceride-rich lipoproteins (Grow and Fried, (1978) J. Biol. Chem., 253, 1834-1841; Lagocki and Scanu, (1980) J. Biol. Chem., 255, 3701-3706; Schaefer et al., J. Lipid Res., (1982) 23, 1259-1273].

Generally, the total cholesterol/HDL-cholesterol (i.e., TC/HDL) ratio can represent a useful predictor as to the risk of an individual in developing a condition, such as atherosclerosis, metabolic syndrome, heart disease or stroke. The current classification of plasma lipid levels is shown in Table B, although these classifications are subject to change with the analysis of newer risk data:

TABLE B
CLASSIFICATION OF PLASMA LIPID LEVELS

| | | | |
|---|---|---|---|
| TOTAL | <200 | mg/dl | Desirable |
| CHOLESTEROL | 200-239 | mg/dl | Borderline High |
| | >240 | mg/dl | High |
| HDL- | <40 | mg/dl | Low (Men) |
| CHOLESTEROL | <50 | mg/dl | Low (Women) |
| | >60 | mg/dl | High |

From: 2001 National Cholesterol Education Program Guidelines

Accordingly, the recommended total cholesterol/HDL-C (i.e., TC/HDL) ratio indicates that a ratio of less than or equal to 3.5 is ideal and a ratio of greater than 4.5 is considered “at risk.” The value of determining the TC/HDL ratio is clearly evident in the circumstance where an individual presents with “normal” LDL and total cholesterol but possesses low HDL-cholesterol. Based on LDL and total cholesterol the individual may not qualify for treatment, however, when factoring in the HDL-cholesterol level, a more accurate risk assessment can be obtained. Thus, if the individual's level of HDL-cholesterol is such that the ratio is greater than 4.5 then therapeutic or prophylactic intervention can be warranted.

Regarding LDL-cholesterol levels, the American Heart Association considers an LDL-cholesterol level of less than 100 mg/dL as optimal, 100-129 mg/dL is near optimal, 130-159 mg/dL is borderline high, 160-189 mg/dL is high and 190 mg/dL is considered a very high level of LDL-cholesterol. Regarding triglyceride levels, the American Heart Association considers less than 150 mg/L as normal, 150-199 mg/dL is borderline-high, 200-499 mg/dL is high and 500 mg/dL is considered a very high level of triglycerides.

The amount of a niacin modulator required in order to alter the amount of atherosclerosis associated serum lipids will vary with the formulation of the compound and the individual. In particular, the amount of a niacin modulator required to alter the amount of atherosclerosis associated serum lipids can be dependent on, for example, the body weight of the individual, genetic makeup of the individual, or the general health of the individual. Amounts of a niacin modulator that can alter the amount of atherosclerosis associated serum lipids can include, for example, at least 500 mg per day, at least 750 mg per day, at least 1 g per day, at least 1.5 g per day, at least 2 g per day, at least 2.5 g per day, at least 3 g per day, at least 3.5 g per day, at least 4 g per day, at least 4.5 g per day, at least 5 g per day, at least 5.5 g per day, at least 6 g per day, at least 6.5 g per day, at least 7 g per day, at least 7.5 g per day, at least 8 g per day, or more. In one embodiment, said lipid altering amount of a niacin modulator is at least 500 mg per day. In another embodiment, said lipid altering amount of a niacin modulator is 1 to 3 grams per day.

As used herein the term “lipid-associated disorder” means any disorder related to a non-optimal level of an atherosclerosis associated serum lipid, for example, LDL-cholesterol, VLDL-cholesterol, HDL-cholesterol, Lp(a), or triglycerides in a subject. Therefore, a lipid-associated disorder can be, for example, an elevated level of LDL-cholesterol, a reduced level of HDL-cholesterol, or disorders that are caused, at least in part, by a non-optimal level of an atherosclerosis associated serum lipid such as atherosclerosis, metabolic syndrome, heart attack (myocardial infarction), or stroke. Optimal levels of atherosclerosis associated serum lipids were discussed above and non-optimal levels of these lipids or less than optimal ratios of these lipids are considered to be lipid-associated disorders.

Atherosclerosis refers to a form of vascular disease characterized by the deposition of atheromatous plaques containing cholesterol and lipids on the innermost layer of the walls of large and medium-sized arteries. Atherosclerosis encompasses vascular diseases and conditions that are recognized and understood by physicians practicing in the relevant fields of medicine. Atherosclerotic cardiovascular disease, including restenosis following revascularization procedures, coronary heart disease, cerebrovascular disease including multi-infarct dementia, and peripheral vessel disease including erectile dysfunction, are all clinical manifestations of atherosclerosis and are therefore encompassed the term atherosclerosis.

Dyslipidemia is a general term for abnormal concentrations of serum lipids such as HDL (low), LDL (high), VLDL (high), triglycerides (high), lipoprotein (a) (high), free fatty acids (high) and other serum lipids, or combinations thereof. For example, an individual with dyslipidemia can have a high level of total cholesterol compared with the optimum level (hypercholesterolemia). In addition, for example, an individual with dyslipidemia can have a high level of a serum lipid such as low-density lipoprotein (LDL) or triglycerides (hypertriglyceridemia). Further, for example, an individual with dyslipidema can have a low level of a serum lipid such as high-density lipoprotein (HDL). An individual with dyslipidemia can have alterations in the level of one or more serum lipids such as, for example, total cholesterol, LDL, triglycerides, or HDL.

Hyperlipidemia, is a general term for elevated concentrations of any or all of the lipids in the plasma such as cholesterol, triglycerides and lipoproteins, is a lipid-associated disorder. Hyperlipidemia can be acquired or can be congenital. Specific forms of hyperlipidemia can include, for example, hypercholesteremia, familial dysbetalipoproteinemia, diabetic dyslipidemia, nephrotic dyslipidemia and familial combined hyperlipidemia. Hypercholesteremia is characterized by an elevation in serum low density lipoprotein-cholesterol and serum total cholesterol. Familial dysbetalipoproteinemia, also known as Type III hyperlipidemia, is characterized by an accumulation of very low density lipoprotein-cholesterol (VLDL-cholesterol) particles called beta-VLDLs in the serum. Also associated with this condition, is a replacement of normal apolipoprotein E3 with abnormal isoform apolipoprotein E2. Diabetic dyslipidemia is characterized by multiple lipoprotein abnormalities, such as an overproduction of VLDL-cholesterol, abnormal VLDL triglyceride lipolysis, reduced LDL-cholesterol receptor activity and, on occasion, Type III hyperlipidemia. Nephrotic dyslipidemia is difficult to treat and frequently includes hypercholesteremia and hypertriglyceridemia. Familial combined hyperlipidemia is characterized by multiple phenotypes of hyperlipidemia, i.e., Type IIa, IIb, IV, V or hyperapobetalipoproteinemia.

Disorders that are caused, at least in part, by a non-optimal level of an atherosclerosis associated serum lipid are included in the definition of a lipid-associated disorder. Such disorders include, for example, coronary artery disease (CAD) or coronary heart disease, congestive heart failure, angina, aneurysm, ischemic heart disease, myocardial infarction and stroke. A lipid-associated disorder can include heart disease such as coronary heart disease, which are disorders comprising a narrowing of the small blood vessels that supply blood to the heart and congestive heart failure where the heart loses its ability to pump blood efficiently. A lipid-associated disorder can include a disorder caused by reduced blood flow to a tissue or organ due to partial or complete blockage of a blood vessel. Such disorders include, for example, angina, ischemic heart disease, myocardial infarction and stroke. A lipid-associated disorder can include a disorder caused by weakened blood vessels such as, for example, an aneurysm, which is a weakened area in a blood vessel often caused by atherosclerosis.

Heart disease includes, but is not limited to, cardiac insufficiency, coronary insufficiency, coronary artery disease, and high blood pressure (hypertension). Peripheral vascular disease refers to diseases of blood vessels outside the heart and brain. Organic peripheral vascular diseases are caused by structural changes in the blood vessels, such as inflammation and tissue damage. Peripheral artery disease is an example. Peripheral artery disease (PAD) is a condition similar to coronary artery disease and carotid artery disease. In PAD, fatty deposits build up along artery walls and affect blood circulation, mainly in arteries leading to the legs and feet. In its early stages a common symptom is cramping or fatigue in the legs and buttocks during activity. Such cramping subsides when the person stands still. This is called “intermittent claudication.” People with PAD have a higher risk of death from stroke and heart attack, due to the risk of blood clots.

Metabolic syndrome, also called Syndrome X, is characterized by a group of metabolic risk factors in one person. They include: central obesity (excessive fat tissue in and around the abdomen), atherogenic dyslipidemia (serum lipid disorders—mainly high triglycerides and low HDL cholesterol), raised blood pressure (130/85 mmHg or higher), insulin resistance or glucose intolerance, prothrombotic state (e.g., high fibrinogen or plasminogen activator inhibitor in the blood), and proinflammatory state (e.g., elevated high-sensitivity C-reactive protein in the blood).

The methods and compositions of the invention can be used to prevent or treat a lipid-associated disorder in a subject. When used to prevent a lipid-associated disorder, the subject can have optimal levels of lipids but may be at risk for a lipid-associated disorder for another reason, for example, a family history of a lipid-associated disorder. The methods and compositions of the invention can be used prophylactically to prevent a lipid-associated disorder in a subject of any age, for example, in a child or adult with obesity or diabetes which are risk factors for developing a lipid-associated disorder.

The invention provides a method for decreasing LDL levels in a subject in need thereof, comprising administering to said subject an effective amount of a niacin receptor modulator with reduced flushing effect compared to niacin or a niacin analog, wherein said modulator with reduced flushing effect compared to niacin or a niacin analog is identified according to the method of: determining the MAP kinase activity of said modulator, wherein a decrease in MAP kinase activity induced by said modulator compared to MAP kinase activity induced by niacin or a niacin analog indicates that said modulator has reduced flushing effect when compared to niacin or a niacin analog. The invention discloses a method for decreasing LDL levels in a subject in need thereof, comprising administering to said subject an effective amount of a niacin receptor modulator with reduced flushing effect compared to niacin or a niacin analog, wherein said modulator with reduced flushing effect compared to niacin or a niacin analog is identified according to the method of: a) identifying a niacin receptor modulator, and b) determining the MAP kinase activity of said modulator, wherein a decrease in MAP kinase activity induced by said modulator compared to MAP kinase activity induced by niacin or a niacin analog indicates that said modulator has reduced flushing effect when compared to niacin or a niacin analog.

The invention also provides a method for decreasing triglyceride levels in a subject in need thereof, comprising administering to said subject an effective amount of a niacin receptor modulator with reduced flushing effect compared to niacin or a niacin analog, wherein said modulator with reduced flushing effect compared to niacin or a niacin analog is identified according to the method of: determining the MAP kinase activity of said modulator, wherein a decrease in MAP kinase activity induced by said modulator compared to MAP kinase activity induced by niacin or a niacin analog indicates that said modulator has reduced flushing effect when compared to niacin or a niacin analog. The invention also discloses a method for decreasing triglyceride levels in a subject in need thereof, comprising administering to said subject an effective amount of a niacin receptor modulator with reduced flushing effect compared to niacin or a niacin analog, wherein said modulator with reduced flushing effect compared to niacin or a niacin analog is identified according to the method of: a) identifying a niacin receptor modulator, and b) determining the MAP kinase activity of said modulator, wherein a decrease in MAP kinase activity induced by said modulator compared to MAP kinase activity induced by niacin or a niacin analog indicates that said modulator has reduced flushing effect when compared to niacin or a niacin analog.

The invention further provides a method for increasing HDL levels in a subject in need thereof, comprising administering to said subject an effective amount of a niacin receptor modulator with reduced flushing effect compared to niacin or a niacin analog, wherein said modulator with reduced flushing effect compared to niacin or a niacin analog is identified according to the method of: determining the MAP kinase activity of said modulator, wherein a decrease in MAP kinase activity induced by said modulator compared to MAP kinase activity induced by niacin or a niacin analog indicates that said modulator has reduced flushing effect when compared to niacin or a niacin analog. The invention further discloses a method for increasing HDL levels in a subject in need thereof, comprising administering to said subject an effective amount of a niacin receptor modulator with reduced flushing effect compared to niacin or a niacin analog, wherein said modulator with reduced flushing effect compared to niacin or a niacin analog is identified according to the method of: a) identifying a niacin receptor modulator, and b) determining the MAP kinase activity of said modulator, wherein a decrease in MAP kinase activity induced by said modulator compared to MAP kinase activity induced by niacin or a niacin analog indicates that said modulator has reduced flushing effect when compared to niacin or a niacin analog.

The invention provides a method for the manufacture of a medicament comprising a niacin receptor modulator with reduced flushing effect compared to niacin or a niacin analog, wherein said modulator with reduced flushing effect compared to niacin or a niacin analog is identified according to the method of: determining the MAP kinase activity of said modulator, wherein a decrease in MAP kinase activity induced by said modulator compared to MAP kinase activity induced by niacin or a niacin analog indicates that said modulator has reduced flushing effect when compared to niacin or a niacin analog, for use as a lipid altering agent. The invention discloses a method for the manufacture of a medicament comprising a niacin receptor modulator with reduced flushing effect compared to niacin or a niacin analog, wherein said modulator with reduced flushing effect compared to niacin or a niacin analog is identified according to the method of: a) identifying a niacin receptor modulator, and b) determining the MAP kinase activity of said modulator, wherein a decrease in MAP kinase activity induced by said modulator compared to MAP kinase activity induced by niacin or a niacin analog indicates that said modulator has reduced flushing effect when compared to niacin or a niacin analog, for use as a lipid altering agent.

The invention also provides a method for the manufacture of a medicament comprising a niacin receptor modulator with reduced flushing effect compared to niacin or a niacin analog, wherein said modulator with reduced flushing effect compared to niacin or a niacin analog is identified according to the method of: determining the MAP kinase activity of said modulator, wherein a decrease in MAP kinase activity induced by said modulator compared to MAP kinase activity induced by niacin or a niacin analog indicates that said modulator has reduced flushing effect when compared to niacin or a niacin analog, for use in the treatment of a lipid-associated disorder. The invention also discloses a method for the manufacture of a medicament comprising a niacin receptor modulator with reduced flushing effect compared to niacin or a niacin analog, wherein said modulator with reduced flushing effect compared to niacin or a niacin analog is identified according to the method of: a) identifying a niacin receptor modulator, and b) determining the MAP kinase activity of said modulator, wherein a decrease in MAP kinase activity induced by said modulator compared to MAP kinase activity induced by niacin or a niacin analog indicates that said modulator has reduced flushing effect when compared to niacin or a niacin analog, for use in the treatment of a lipid-associated disorder.

The invention also discloses methods for combination therapy which includes another therapeutic compound or compounds in addition to a niacin receptor modulator. Other therapeutic compounds can include, for example, compounds that can be used to further reduce flushing or compounds that can be used to further lower the amount of atherosclerosis associated serum lipids in a subject.

Therapeutic compounds that can be combined with a niacin receptor modulator can include, for example, compounds that reduce prostaglandin synthesis, such as PGD₂ synthesis. Such compounds can include, for example, specific PGD₂ antagonists, or more general agents such as non-steroidal anti-inflammatory drugs (NSAIDs). Examples of NSAIDS include: aspirin, salicylate salts, ibuprofen, indomethacin, naproxen, sodium naproxen, ketoprofen, fenoprofen, oxaprozin, sulindac, flurbiprofen, etodolac, diclofenac, ketorolac, tolmetin, nabumetone, suprofen, benoxaprofen, carprofen, aclofenac, fenclofenac, zomepirac, meclofenamate, mefanamic acid, oxyphenbutazone, phenylbutazone and piroxicam. In addition, to combinations with COX-1 inhibitors, the therapeutic compounds can be combined with selective COX-2 inhibitors such as Celecoxib or Rofecoxib.

Therapeutic compounds that can be combined with a niacin receptor modulator can include, for example, compounds that lower the amount of atherosclerosis associated serum lipids in subjects. Such compounds include, for example, a α-glucosidase inhibitor, aldose reductase inhibitor, biguanide, HMG-CoA reductase inhibitor, squalene synthesis inhibitor, fibrate, LDL catabolism enhancer, angiotensin converting enzyme (ACE) inhibitor, insulin secretion enhancer and thiazolidinedione.

α-Glucosidase inhibitors belong to the class of drugs which competitively inhibit digestive enzymes such as α-amylase, maltase, α-dextrinase, sucrase, etc. in the pancreas and or small intestine. The reversible inhibition by α-glucosidase inhibitors retard, diminish or otherwise reduce blood glucose levels by delaying the digestion of starch and sugars. Some representative examples of α-glucosidase inhibitors include acarbose, N-(1,3-dihydroxy-2-propyl)valiolamine (generic name; voglibose), miglitol, and α-glucosidase inhibitors known in the art.

Aldose reductase inhibitors are drugs which inhibit the first-stage rate-limiting enzyme in the polyol pathway. Examples of the aldose reductase inhibitors include tolurestat; epalrestat; 3,4-dihydro-2,8-diisopropyl-3-thioxo-2H-1,4-benzoxazine-4-acetic acid; 2,7-difluorospiro(9H-fluorene-9,4′-imidazolidine)-2′,5′-dione (generic name: imirestat); 3-[(4-bromo-2-fluorophenyl)methy]-7-chloro-3,4-dihydro-2,4-dioxo-1(2H)-quinazoline acetic acid (generic name: zenarestat); 6-fluoro-2,3-dihydro-2′,5′-dioxo-spiro[4H-1-benzopyran-4,4′-imidazolidine]-2-carboxamide (SNK-860); zopolrestat; sorbinil; and 1-[(3-bromo-2-benzofuranyl)sulfonyl]-2,4-imidazolidinedione (M-16209), and aldose reductase inhibitors known in the art.

The biguanides are a class of drugs that stimulate anaerobic glycolysis, increase the sensitivity to insulin in the peripheral tissues, inhibit glucose absorption from the intestine, suppress of hepatic gluconeogenesis, and inhibit fatty acid oxidation. Examples of biguanides include phenformin, metformin, buformin, and biguanides known in the art.

Statin compounds belong to a class of drugs that lower blood cholesterol levels by inhibiting hydroxymethylglutalyl CoA (HMG-CoA) reductase. HMG-CoA reductase is the rate-limiting enzyme in cholesterol biosynthesis. A statin that inhibits this reductase lowers serum LDL concentrations by upregulating the activity of LDL receptors and responsible for clearing LDL from the blood. Examples of the statin compounds include rosuvastatin, pravastatin and its sodium salt, simvastatin, lovastatin, atorvastatin, fluvastatin, cerivastatin, and HMG-CoA reductase inhibitors known in the art.

Squalene synthesis inhibitors belong to a class of drugs that lower blood cholesterol levels by inhibiting synthesis of squalene. Examples of the squalene synthesis inhibitors include (S)-α-[Bis[2,2-dimethyl-1-oxopropoxy)methoxy]phosphinyl]-3-phenoxybenzenebutanesulfonic acid, mono potassium salt (BMS-188494) and squalene synthesis inhibitors known in the art.

Fibrate compounds belong to a class of drugs that lower blood cholesterol levels by inhibiting synthesis and secretion of triglycerides in the liver and activating a lipoprotein lipase. Fibrates have been known to activate peroxisome proliferators-activated receptors and induce lipoprotein lipase expression. Examples of fibrate compounds include bezafibrate, beclobrate, binifibrate, ciplofibrate, clinofibrate, clofibrate, clofibric acid, etofibrate, fenofibrate, gemfibrozil, nicofibrate, pirifibrate, ronifibrate, simfibrate, theofibrate, and fibrates known in the art.

LDL (low-density lipoprotein) catabolism enhancers belong to a class of drugs that lower blood cholesterol levels by increasing the number of LDL receptors, examples include LDL catabolism enhancers known in the art.

Angiotensin converting enzyme (ACE) inhibitors belong to the class of drugs that partially lower blood glucose levels as well as lowering blood pressure by inhibiting angiotensin converting enzymes. Examples of the angiotensin converting enzyme inhibitors include captopril, enalapril, alacepril, delapril; ramipril, lisinopril, imidapril, benazepril, ceronapril, cilazapril, enalaprilat, fosinopril, moveltopril, perindopril, quinapril, spirapril, temocapril, trandolapril, and angiotensin converting enzyme inhibitors known in the art.

The invention provides a method for preventing or treating a lipid-associated disorder in a subject, comprising administering to said subject an effective lipid altering amount of a niacin receptor modulator with reduced flushing effect compared to niacin or a niacin analog identified by the method of: determining the MAP kinase activity of said modulator, further comprising administering to said subject an effective amount of an agent used for the treatment of obesity or diabetes in combination with an effective amount of niacin receptor modulator with reduced flushing effect compared to niacin or a niacin analog.

Lipase inhibitors include, for example, anti-obesity compounds such as Orlistat (XENICAL™). Orlistat inhibits fat absorption directly but also tends to produce a high incidence of unpleasant gastric side-effects such as diarrhea and flatulence.

Another class of anti-obesity drugs includes serotonin and/or noradrenaline releasers or reuptake inhibitors. For example, sibutramine (Meridia™) is a mixed 5-HT/noradrenaline reuptake inhibitor. The main side effect of sibutramine can be an increase in blood pressure and heart rate in some patients. The serotonin releaser/reuptake inhibitors fenfluramine (Pondimin™) and dexfenfluramine (Redux™) have been reported to decrease food intake and body weight over a prolonged period (greater than 6 months). However, both products were withdrawn from use after reports of preliminary evidence of heart valve abnormalities associated with their use.

Insulin secretion enhancers belong to the class of drugs having the property to promote secretion of insulin from pancreatic β cells. Examples of the insulin secretion enhancers include sulfonylureas (SU). The sulfonylureas (SU) are drugs which promote secretion of insulin from pancreatic β cells by transmitting signals of insulin secretion via SU receptors in the cell membranes. Examples of the sulfonylureas include tolbutamide; chlorpropamide; tolazamide; acetohexamide; 4-chloro-N-[(1-pyrrolidinylamino) carbonyl]-benzenesulfonamide (generic name: glycopyramide) or its ammonium salt; glibenclamide (glyburide); gliclazide; 1-butyl-3-metanilylurea; carbutamide; glibonumide; glipizide; gliquidone; glisoxepid; glybuthiazole; glibuzole; glyhexamide; glymidine; glypinamide; phenbutamide; tolcyclamide, glimepiride, and other insulin secretion enhancers known in the art. Other insulin secretion enhancers include N-[[4-(1-methylethyl)cyclohexyl)carbonyl]-D-phenylalanine (Nateglinide); calcium (2S)-2-benzyl-3-(cis-hexahydro-2-isoindolinylcarbonyl)propionate dihydrate (Mitiglinide, KAD-1229); and other insulin secretion enhancers known in the art.

Thiazolidinediones belong to the class of drugs more commonly known as TZDs. Examples of thiazolidinediones include rosiglitazone, pioglitazone, and thiazolidinediones known in the art.

In addition, a niacin receptor modulator can be administered to a subject, for example, in order to prevent or treat a niacin-responsive disorder. A niacin-responsive disorder is a disorder or disease that can be prevented or treated by a receptor modulator. A niacin-responsive disorder can include, for example, a lipid-associated disorder as described herein. For example, a lipid-associated disorder can be a low amount of high density lipoprotein (HDL)-cholesterol, an elevated amount of low density lipoprotein (LDL)-cholesterol, an elevated amount of triglycerides, or a disorder that is caused, at least in part, by a non-optimal level of an atherosclerosis associated serum lipid such as atherosclerosis, heart disease or stroke.

Another example of a niacin responsive disorder is dysmenorrhea or painful menstruation. In one report, a group of 80 women suffering from painful menstrual cramps were supplemented with 100 mg of niacin twice daily, beginning 7 to 10 days before the onset of menses and then every 2 to 3 hours during heavy cramps [Hudgins, (1952) Am Pract Dig Treat 3:892-893; Hudgins (1954) West J Surg Obstet Gynecol 62:610-611]. About 90% of subjects experienced significant relief. The dosage required during heavy cramping (100 mg every 2 to 3 hours) is high enough to cause flushing in some women. In this case, the methods and compositions of the invention can be of use to treat a niacin-responsive disorder without the flushing side effect.

The present invention discloses kits for use by a consumer to prevent or treat a lipid-associated disorder. A kit can comprise a pharmaceutical composition of the invention and instructions describing a method of using the pharmaceutical composition to prevent or treat a lipid-associated disorder. For example, a kit can contain at least one dosage unit of a niacin receptor modulator with reduced flushing effect compared to niacin or a niacin analog. In addition, a kit can include other therapeutic agents used in combination with the compositions of the invention.

The compositions of the invention can be administrated in a wide variety of oral, topical or parenteral dosage forms. It will be obvious to those skilled in the art that the dosage forms can comprise, as the active component, either a compound of the invention or a pharmaceutically acceptable salt of a compound of the invention.

The dosage of active ingredient, or an active salt or derivative thereof, required for use in prophylaxis or treatment will vary not only with the particular salt selected but also with the route of administration, the nature of the condition being treated and the age and condition of the individual and will ultimately be at the discretion of the attendant physician or clinician. In general, one skilled in the art understands how to extrapolate in vivo data obtained in a model system, typically an animal model, to another, such as a human. In some circumstances, these extrapolations can merely be based on the weight of the animal model in comparison to another, such as a mammal, preferably a human, however, more often, these extrapolations are not simply based on weights, but rather incorporate a variety of factors. Representative factors include the type, age, weight, sex, diet and medical condition of the individual, the severity of the disease, the route of administration, pharmacological considerations such as the activity, efficacy, pharmacokinetic and toxicology profiles of the particular compound employed, whether a drug delivery system is utilized, on whether an acute or chronic disease state is being treated or prophylaxis is conducted or on whether combination therapy is used. The dosage regimen for preventing or treating a disease condition with the compounds and/or compositions of this invention is selected in accordance with a variety factors as cited above. Thus, the actual dosage regimen employed can vary widely and therefore can deviate from a preferred dosage regimen and one skilled in the art will recognize that dosage and dosage regimen outside these typical ranges can be tested and, where appropriate, can be used in the methods of this invention.

The desired dose can conveniently be presented in a single dose or as divided doses administered at appropriate intervals, for example, as two, three, four or more sub-doses per day. The sub-dose itself may be further divided, for example, into a number of discrete loosely spaced administrations. The daily dose can be divided, especially when relatively large amounts are administered as deemed appropriate, into several, for example 2, 3 or 4, part administrations. If appropriate, depending on individual behavior, it can be necessary to deviate upward or downward from the daily dose indicated.

A kit can include a container for containing a pharmaceutical composition of the invention and can also include divided containers such as a divided bottle or a divided foil packet. The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. The container employed can depend on the exact dosage form involved, for example a conventional cardboard box would not generally be used to hold a liquid suspension. It is feasible that more than one container can be used together in a single package to market a single dosage form. For example, tablets may be contained in a bottle, which is in turn contained within a box.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process, recesses are formed in the plastic foil. The recesses have the size and shape of individual tablets or capsules to be packed or may have the size and shape to accommodate multiple tablets and/or capsules to be packed. Next, the tablets or capsules are placed in the recesses accordingly and the sheet of relatively stiff material is sealed against the plastic foil at the face of the foil which is opposite from the direction in which the recesses were formed. As a result, the tablets or capsules are individually sealed or collectively sealed, as desired, in the recesses between the plastic foil and the sheet. Generally, the strength of the sheet is such that the tablets or capsules can be removed from the blister pack by manually applying pressure on the recesses whereby an opening is formed in the sheet at the place of the recess. The tablet or capsule can then be removed via said opening.

It can be desirable to provide a written memory aid, where the written memory aid is of the type containing information and/or instructions for the physician, pharmacist or subject, for example, in the form of numbers next to the tablets or capsules whereby the numbers correspond with the days of the regimen which the tablets or capsules so specified should be ingested or a card which contains the same type of information. Another example of such a memory aid is a calendar printed on the card for example, as follows “First Week, Monday, Tuesday,” . . . etc. . . . “Second Week, Monday, Tuesday” etc. Other variations of memory aids will be readily apparent.

Another specific embodiment of a kit is a dispenser designed to dispense the daily doses one at a time. The dispenser can be equipped with a memory-aid, so as to further facilitate compliance with the regimen. An example of such a memory-aid is a mechanical counter which indicates the number of daily doses that has been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

One aspect of the present invention pertains to a niacin receptor modulator as described herein, for use in a method of treatment of the human or animal body by therapy.

Another aspect of the present invention pertains to a niacin receptor modulator with reduced flushing effect compared to niacin or a niacin analog, as described herein, for use in a method of treatment of a lipid associated disorder, of the human or animal body by therapy. Another aspect of the present invention pertains to a method for the treatment of a lipid associated disorder comprising administering to a subject suffering from said condition a therapeutically-effective amount of a niacin receptor modulator with reduced flushing effect compared to niacin or a niacin analog, as described herein, preferably in the form of a pharmaceutical composition.

One aspect of the present invention pertains to a method for the treatment of a lipid-associated disorder comprising administering to a subject suffering from said condition a therapeutically-effective amount of a niacin receptor modulator with reduced flushing effect compared to niacin or a niacin analog, as described herein, preferably in the form of a pharmaceutical composition. Another aspect of the present invention pertains to a niacin receptor modulator with reduced flushing effect compared to niacin or a niacin analog, as described herein, for use in a method of treatment of a lipid-associated disorder of the human or animal body by therapy.

Compounds shown in the application are either commercially available or can be synthesized using methods know in the art, for example, as described in van Herk et al., J. Med Chem. 46:3945-3951 (2003) or PCT/US2004/038920.

### EXAMPLES

The following Examples are provided for illustrative purposes and not as a means of limitation. One of ordinary skill in the art would be able to design equivalent assays and methods based on the disclosure herein, all of which form part of the present invention.

### Example 1

### Antibody Based Assays to Determine Induction of MAP Kinase

This example shows an ELISA and Western Blot assay that can be used to determine MAP kinase activity induced by niacin (see FIG. 1) or niacin and a niacin receptor modulator Compound 8 (see FIG. 2).

### MAP Kinase ELISA:

A kit from Biosource (phosphoERK1/2 pT185pY187 ELISA, Catalog #KHO-0091) was used according to the following protocol.

The cells were serum-starved overnight prior to stimulating the cells with compound.

### 1. Compound Preparation and Cell Treatment:

A. Compounds were dissolved in DMSO. Do not go over 1% DMSO because higher DMSO concentrations will stress the cells and activate MAPK. PMA (100 ng/ml) was used as a positive control.
B. Cell dishes were taken out of the incubator and placed on a rocker set to gentle rocking (set speed at 4). Compound was carefully added and cells were returned to the incubator to incubate for 5 min. At 4.5 min, the medium was aspirated from dishes in the order that the compound was added. Then 2 ml cold PBS was added and excess medium was removed by gentle swirling. The PBS was aspirated and 1 ml of PBS was added (1 ml for confluent 6 cm dish).

### 2. Cell Collection and Extraction: On Ice

A. Cells were scraped from dish with a rubber policeman and transferred to a microfuge tube, then centrifuged at 3000 rpm at 4° C. for 5 minutes.
B. The PBS was aspirated and cell pellet lysed in Cell Extraction Buffer (0.1% SDS) (250-300 μl for confluent 6 cm dish) for 30 min. on ice with vortexing at 10 min intervals.
C. The mixture was then centrifuged at max speed (16,000×G) for 15 min. at 4° C.
D. Clarified lysates were transferred to new microfuge tubes and protein concentration measure. To measure protein, samples were diluted with Cell Extraction Buffer to a concentration of 1 mg/ml then boiled for 5 min. After cooling, they were centrifuged at max speed for 5 min at room temperature. Lysates were diluted 1:10 with Standard Diluent Buffer to a concentration of 0.1 mg/ml (0.01% SDS final) and loaded 100 μl in duplicate to sample wells (10 μg/well). Lysates can be stored at −80° C.

### 3. Reagent Preparation and Storage:

A. Reconstitution and dilution of phospho ERK1/2 standard:
1. Phospho ERK1/2 standard was reconstituted with 1.2 ml Standard Diluent Buffer, mixed gently and allowed to sit for 10 min. to ensure complete reconstitution. This stock is 100 U/ml.
2. In duplicate, 125 μl of Standard Diluent Buffer was added to wells B-H of master plate (not ELISA plate). 250 μl of 100 U/ml stock was added to well A.
3. Serial dilutions (1:2) were made by transferring 125 μl of 100 U/m in well A to well B, mixing and transferring 125 μl of well B to well C and so on until well G. Well H was not diluted (0 U/ml).
B. Storage and final dilution of αRabbit IgG HRP:
1. αRabbit IgG HRP concentrate was brought to room temperature and gently mixed. Then Mix 10 μl of concentrate was mixed with 1 ml of HRP Diluent for each 8-well strip used in assay.
C. Dilution of wash buffer:
1. The 25× wash buffer concentrate was brought to RT and mixed to ensure full reconstitution. Wash buffer concentrate was diluted with deionized water (40 ml 25×/960 ml H₂O).
4. Assay method: Procedure and calculations:

### Standard and Sample Application:

A. All reagents were at room temperature and mixed before use.
B. Microtiter plates were at room temperature before opening foil bags. The number of 8 well strips needed for assay was determined and bag was sealed and returned to 4° C.
C. 100 μl of standard (prepared in 3A2) was added in duplicate (2 8-well strips).
D. Two wells were left empty for chromogen blank.
E. 100 μl of samples were added in duplicate to sample wells.
F. Plate was covered with plate cover and tapped gently on side of plate to mix.
G. Plates were incubated at room temperature for 2 hours. (The plate may be incubated overnight at 4° C.).

### Washes.

A. Liquid from wells was aspirated with aspirator.

B. The wells were filled with 200 μl of diluted wash buffer. After incubation for 30 sec. the liquid was aspirated. This was repeated 4 times.

### Detection Antibody.

A. 100 μl of αphosphoERK1/2 solution was pipetted into each well except the chromogen blanks. The cover was replaced and tapped gently to mix.

B. Incubation occurred for 1 hour at room temperature.

### Washes.

A. The wells were washed 4 times as above. αRabbit IgG HRP.

A. 100 μl of αRabbit IgG HRP working solution was added to each well except the chromogen blanks. The cover was replaced and tapped gently to mix.
B. Incubation occurred for 30 min at room temperature.

### Washes.

A. The wells were washed 4 times as above.

### Chromogen.

A. 100 μl of Stabilized Chromogen was added to each well.
B. Incubation occurred for 20 min. at room temperature in dark. (Do not use foil or metal)

### Stop Solution.

A. 100 μl of Stop Solution was added to each well and tapped to mix the plate.
Reading the plate.
A. The plate was read at an absorbance of 450 nm.

### Cell Extraction Buffer:

| | | |
|---|---|---|
| 10 mM Tris pH 7.4 | 5 | ml (1 M) |
| 100 mM NaCl | 10 | ml (5 M) |
| 1 mM EDTA pH 8.0 | 1 | ml (0.5 M) |
| 20 mM Na4P2O7 | 100 | ml (100 mM) |
| 1% TX-100 | 5 | ml (100%) |
| 10% glycerol | 50 | ml (100%) |
| 0.1% SDS | 5 | ml (10%) |
| 0.5% Deoxycholate | 2.5 | g |
| | 500 | ml final volume |

Add fresh:

| | | |
|---|---|---|
| 2 mM Na3VO4 | 1 | ml (100 mM) |
| 1 mM PMSF | 250 | ul (200 mM) |
| 25 ug/ml Leupeptin | 125 | ul (10 ug/ul) |
| 25 ug/ml Aprotinin | 125 | ul (10 ug/ml) |
| | 50 | ml final volume |

MAP kinase Western Blot:

### 1. Sample Preparation:

A. The following steps were done on ice. Medium was aspirated off cells and cells rinsed with PBS.
B. Cells were harvested in appropriate volume of 1% NP-40 lysis buffer (volume depends on dish size, cell density, etc). Typically, 500 μl was used for a confluent 6 cm dish.
C. Lysate was transferred into a microfuge tube. The tube was vortexed and incubated on ice for 30 min. the centrifuged at max speed, 4° C. for 10 min.

### 2. Protein Assay:

A. Stock protein standard BSA was prepared @ 1.41 μg/μl in water.
B. 14.2 μl stock standard was added to 485.8 μl water=40 μg/ml.
C. 200 μl of 40 μg/ml standard was added to well 9A and 9B.
D. 100 μl of water was added to 1-8, rows A and B.
E. A serial dilution was performed by adding 100 μl of 40 μg/ml to the 20 μg/ml well, mixing and transferring 100 μl into next well until you reach the 0.31 μg/ml well. The last 100 μl from the 0.31 ug/ml well was discarded.
F. 99 μl of water was added to the wells designated for unknowns.
G. 1 μl of unknown sample was added to wells in triplicate.
H. 25 μl of 5× Bradford dye reagent was added to standards and unknowns.
I. Incubation occurred at room temperature for at least 5 minutes.
J. Absorbance was read at 595λ.

### 3. Sample Dilution and Preparation for Loading:

A. Samples were diluted to a final concentration of 1 μg/μl with water or lysis buffer.
B. 5× Laemmli sample buffer was added, and samples were vortexed and boiled for 5 min.

### 4. Set up for NOVEX Gels:

A. White adhesive strip at the bottom of gel was pulled off.
B. The comb was gently pulled out.
C. Gels were rinsed with water and placed in gel box.
D. The inner reservoir was filled with Running buffer and the outer reservoir filled above the gel opening (where the white strip was).
E. The wells were flushed with a syringe.

### 5. Loading Samples:

A. Samples were loaded being careful not to spill over into the adjacent wells.
B. Standard markers were loaded and Empty wells loaded with 1× sample buffer.

### 6. Running the Gel:

A. The gels were run at 150V for 1.5 hr.
7. Transfer to Nitrocellulose (0.2 μm pore size):
A. 1× transfer buffer was prepared.
B. Gel, sponges, Whatman paper and nitrocellulose membranes were soaked in transfer buffer.
C. Layering was done in the following order: positive electrode, sponges, membrane, gel, sponges, negative electrode.
D. Outer and inner chamber was filled with transfer buffer.
E. The transfer occurred at 25 V for 1.5 hour.

### 8. Blocking of Membrane:

A. Transfer rig was dismantled.
B. Nitrocellulose membrane was placed in BLOCKO and incubated overnight at 4° C. on a rocker.

### 9. Primary Antibody:

A. Membrane was washed 1×10 min. with TBS/tween on a rocker.
B. Primary antibody was diluted in BLOCKO
C. Incubation occurred on a rocker for 2 hr at room temperature.

### 10. Secondary Antibody:

A. Membrane was washed 2×15 min with TBS/tween on a rocker.
B. Secondary antibody was diluted in TBS/tween.
C. Incubation occurred on a rocker for 1 hr at room temperature.

### 11. Detection:

A. Membrane was washed 3×15 min with TBS/tween on a rocker.
B. Membrane was rinsed once with water.
C. Chemiluminescent detection reagent was added (10 ml ECL reagent+5 ul H₂O₂ (30%) per membrane) and rocked for 2 min.
D. Membrane was placed in plastic sheet protector and excess detection reagent and bubbles were squeezed out.
E. The membrane was exposed to film.
1% NP-40 Lysis buffer:

### 1% NP-40

### 20 nM Tris pH 8.0

### 100 mM NaCl

### 1 mM EDTA

### 1 mM PMSF

### 200 μM Na₃V0₄

### 10 U/ml Aprotinin

10 μg/ml Leupeptin

### 5× Laemmli Sample Buffer:

### 300 mM Tris pH 6.8

### 25% Glycerol

### 10% SDS

### 0.05% Bromophenol Blue

120 ul/ml final volume of stock 2-βME

### SDS-PAGE Running Buffer:

### 14.4 g Glycine

3.03 g Tris base

### 1 g or 5 ml (20%) SDS

q.s. to 1 liter with water

### 10× Transfer Buffer:

0.2 M Tris base

### 1.92 M Glycine

### 1× Transfer Buffer:

100 ml 10× Transfer buffer

### 200 ml MeOH

700 ml water

### 10× TBS:

60.5 g Tris base

### 87.5 g NaCl

q.s to 1 Liter with water

### TBS/Tween:

### 100 ml 10×TBS

900 ml water

### 500 ul Tween20 (100%)

### BLOCKO:

### 4% BSA in TBS/Tween

### Example 2

### Correlation Between MAP Kinase Activity and In Vivo Flushing Effect of Niacin Receptor Modulators

This example shows that compounds with known flushing effects in vivo had higher levels of MAP kinase activity than compounds that were known not to cause significant flushing in vivo (see FIGS. 3 and 4). Note in FIG. 3 that Compound 1 is niacin.

For the table in FIG. 3, in vivo flushing and cAMP was measured essentially as follows. MAP kinase was measured using the ELISA protocol in Example 1.

### Flushing in Mice Using a Laser Dopler:

Male C57B16 mice (˜25 g) were anesthetized using 10 mg/ml/kg Nembutal sodium (Abbott labs). After ten minutes the animal was placed under the laser and the ear was folded back to expose the ventral side. The laser was positioned in the center of the ear and focused to an intensity of 8.4-9.0 V (which is generally ˜4.5 cm above the ear). Baseline readings were recorded for 3 minutes. Data acquisition was initiated with a 15 by 15 image format, auto interval, 60 images and a 20 second time delay with a medium resolution. Test compounds were administered following the 10th image via injection into the peritoneal space. Images 1-10 were considered the animal's baseline and data was normalized to an average of the baseline mean intensities. The Laser Doppler used was Pirimed PimII.

### Cyclic AMP Assay

An Adenylyl Cyclase Activation Kit, 96 well, from Perkin Elmer (catalog no. SMP004B) was used. The cell culture medium for the CHO cells was F-12 Kaighn's Modified Cell Culture Medium with 10% fetal bovine serum, 2 mM L-glutamine, 1 mM sodium pyruvate and 400 μg/ml G418.

### Reagent Preparations

50/50 assay buffer was made (Stimulation Buffer/PBS; note that stimulation buffer is included in the Flashplate kit.)
2% DMSO in PBS was made.
10000 pmol/ml (10 μM) cAMP standard stock solution was made by dissolving one bottle of cAMP powder in 1 mL Stimulation Buffer. A serial dilution as described in the table below was done to create the eight standard concentrations:

| Pipette of | | | | | |
|---|---|---|---|---|---|
| 10 μM cAMP | | Add 2% | | Conc. | |
| stock | From | DMSO in | Into | (pmol/ml) | Final |
| (mls) | tube | PBS (mls) | tube | (nM) | (pmol/well) |
| 1 | Stock | 4 | A | 2000 | 50 |
| 2 | A | 2 | B | 1000 | 25 |
| 2 | B | 2 | C | 500 | 12.5 |
| 2 | C | 3 | D | 200 | 5 |
| 2 | D | 2 | E | 100 | 2.5 |
| 2 | E | 2 | F | 50 | 1.25 |
| 2 | F | 3 | G | 20 | 0.5 |

20 μM forskolin was made by diluting a 10 mM stock (stored frozen) 1:500 in stimulation buffer.
40 μM niacin was made in 2% DMSO/PBS

### Preparation of Serial 5×-Dilutions of Compounds (in 96 Well Plates)

5 μl 20 mM compound was put in 100% DMSO at column #2, one plate can have a maximum of 8 compounds.
245 μl PBS was added to column #2 wells that contain compounds and mixed by up/down pipetting. 200 μl 2% DMSO/PBS was placed in columns (#3 to #11).
50 μl from column #2 was transferred to column #3, mixed as above, and repeated serial dilution to column #11 (changing pipette tips with each dilution). Now column #2 to #11 have compounds ranging from 400 μM to 204.8 pM in 2% DMSO (which is 4× the final assay concentrations of 100 μM to 51.2 pM).

### Harvesting Cells

Culture medium was aspirated from T-185 cell culture flasks. Cells were washed once with 10 mL PBS, and 5 mL per flask of warm (37° C.) Cell Dissociation Solution was added. After cells detached, 5 mL warm (37° C.) 50/50 buffer was added to each flask, and cells were transferred from the flasks to 50 mL conical tubes.
Cells were centrifuged at 1100 rpm at room temperature for 6 minutes. The supernatant was aspirated and cells resuspended in 50/50 assay buffer. Cells were counted and diluted to a density of 1×10⁶ cells/ml with warm (37° C.) 50/50 assay buffer.

### Reagent Addition

200 μL of 2% DMSO/PBS was added to the first 4 rows of column #1, 200 μL of 40 μM niacin to the last 4 rows of column #1, and cAMP standards to column #12 on the compound plates.
25 μL was transferred from compound plates into all wells of three separate assay plates.
50 μl of 50/50 (Stimulation/PBS) was added into column #12 of assay plates.
50 μL of cells was added into columns #1-11, final density was 50,000 cells/well.
25 μl of 20 μM Forskolin was added (final concentration of 5 μM) to all wells containing cells expressing the niacin receptor, and all wells except column #1, rows E-H, since column 1 contained cells expressing a negative control receptor, to which 25 μl of stimulation buffer was added.
Plates were put on shaker for 1 hour at room temperature, the top plates were covered with foil.
50 μl [¹²⁵I]-cAMP was diluted to 11 ml detection buffer (in kit), and 100 μL of diluted tracer was added to each well.
Plates were covered with plastic cover and put on shaker at room temperature for 2 hours.
Plates were counted in the Wallac Microbeta Counter 1450 with the ¹²⁵I FlashPlate Protocol to detect ¹²⁵I.

### Example 3

### Predictive Ability of the MAP Kinase Assay

This example shows that a compound chosen based on a cAMP assay, with no knowledge of its ability to cause flushing in vivo, was tested for its ability to induce MAP kinase compared to niacin. The compound (Compound 11) was found to have reduced ability to induce MAP kinase compared to niacin and so was tested for in vivo flushing activity in mice using the Laser Doppler assay described in Example 2. As predicted by the methods of the invention, the compound did not cause flushing in vivo. Thus, the MAP kinase assay was able to predict whether the compound would cause flushing when tested in vivo. In addition, the compound was tested for its ability to reduce free fatty acids (FFAs).

In this example, the MAP kinase activity was measured using the MAP kinase ELISA described in Example 1. An ELISA assay using CHO cells which expressed either the human niacin receptor (see FIG. 5, upper left panel) or the mouse niacin receptor (see upper right panel) was used. The results of the Laser Doppler flushing assay in mice are shown in FIG. 5, lower right panel. A free fatty acid release assay was performed as described below.

### FFA Assay

Mice were given either vehicle or various doses of Compound 11. After 10 minutes the mice were euthanized and blood was collected. The blood samples were processed and tested for free fatty acid release using the non-esterified fatty-acid (NEFA) assay (the NEFA-C assay kit from Waco Chemicals USA, Richmond, Va.). The NEFA assay was done as per manufacturer suggested protocol. The concentration of free fatty acid measured for the vehicle sample was greater than for Compound 11 which indicates that treatment with Compound 11 caused a reduction of free fatty acid release. Therefore, Compound 11 can be considered an anti-lipolytic compound.

### Example 4

### Compound 12, a Niacin Analog

This example shows an example of a niacin analog (Compound 12) which, for example, can be used in place of niacin in the methods of the invention.

In this example, the MAP kinase activity of Compound 12 was measured using the MAP kinase ELISA described in Example 1. An ELISA assay using CHO cells which expressed either the human niacin receptor (see FIG. 6, left panel) or the mouse niacin receptor (see right panel) was used. As shown in FIG. 6, Compound 12 closely matches the profile of niacin in the MAP kinase activation assay.

### Example 5

### Measurement of Free Fatty Acid Levels in Rats and Lipolysis in Human Adipocytes

This example shows that free fatty acid levels can be measured in rats. This example also shows that free fatty acid levels can be measured in human adipocytes.

### Rat Assay

Catheters are surgically implanted into the jugular veins of male Sprague Dawley rats. Rats are given a few days to recover from catheter implantation surgery and then the following day rats are deprived of food and approximately 16 hours later are given interperitoneal (IP) injections of either vehicle, or niacin [NA] at 15 mg/kg, 30 mg/kg or 45 mg/kg body weight. A niacin analog can be tested in the same manner. Blood is drawn (˜200 ml) at various time points and plasma is isolated following centrifugation. Plasma FFAs are then measured via the NEFA C kit according to manufacturer specifications (Wako Chemicals USA, Inc).

### Human Adipocyte Lipolysis Assay:

Adipocytes are obtained from ZenBio (Research Triangle, N.C.) and the lipolysis assay is performed according to manufacturer's protocol. An elevation of intracellular cAMP levels and concomitant activation of lipolysis via hormone sensitive lipase is accomplished using isoproterenol at concentrations and times determined empirically. Lipolysis is allowed to continue for the desired time in the presence or absence of a compound of interest (for example, niacin or a niacin analog). At least five compound concentrations are tested allowing for non-linear regression analysis and determination of an EC₅₀ value. The percent of glycerol production is measured colorimetrically and is compared to standards (ZenBio).

### Example 6

### Mouse Atherosclerosis Model

Adiponectin-deficient mice generated through knocking out the adiponectin gene have been shown to be predisposed to atherosclerosis and to be insulin resistant. The mice are also a suitable model for ischemic heart disease [Matsuda, M et al. J Biol Chem (2002) July, and references cited therein, the disclosures of which are incorporated herein by reference in their entirety].

Adiponectin knockout mice are housed (7-9 mice/cage) under standard laboratory conditions at 22° C. and 50% relative humidity. The mice are dosed by micro-osmotic pumps, inserted using isoflurane anesthesia, to provide compounds of the invention, saline, or an irrelevant compound to the mice subcutaneously (s.c.). Neointimal thickening and ischemic heart disease are determined for different groups of mice sacrificed at different time intervals. Significant differences between groups (comparing compounds of the invention to saline-treated) are evaluated using Student t-test.

The foregoing mouse model of atherosclerosis is provided by way of illustration and not limitation. By way of further example, Apolipoprotein E-deficient mice have also been shown to be predisposed to atherosclerosis [Plump A S et al., Cell (1992) 71:343-353; the disclosure of which is hereby incorporated by reference in its entirety].

Another model that can be used is that of diet-induced atherosclerosis in C57BL/6J mice, an inbred strain known to be susceptible to diet-induced atherosclerotic lesion formation. This model is well known to persons of ordinary skill in the art [Kamada N et al., Atheroscler Thromb (2001) 8:1-6; Garber D W et al., J Lipid Res (2001) 42:545-52; Smith J D et al., J Intern Med (1997) 242:99-109; the disclosure of each of which is hereby incorporated by reference in its entirety].

### Example 7

### In Vivo Pig Model of HDL-Cholesterol and Atherosclerosis

The utility of a compound of the present invention as a medical agent in the prevention or treatment of a lipid-associated disorder is demonstrated, for example, by the activity of the compound in lowering the ratio of total cholesterol to HDL-cholesterol, in elevating HDL-cholesterol, or in protection from atherosclerosis in an in vivo pig model. Pigs are used as an animal model because they reflect human physiology, especially lipid metabolism, more closely than most other animal models. An illustrative in vivo pig model not intended to be limiting is presented here.

Yorkshire albino pigs (body weight 25.5±4 kg) are fed a saturated fatty acid rich and cholesterol rich (SFA-CHO) diet during 50 days (1 kg chow 35 kg−1 pig weight), composed of standard chow supplemented with 2% cholesterol and 20% beef tallow [Royo T et al., European Journal of Clinical Investigation (2000) 30:843-52; which disclosure is hereby incorporated by reference in its entirety]. Saturated to unsaturated fatty acid ratio is modified from 0.6 in normal pig chow to 1.12 in the SFA-CHO diet. Animals are divided into two groups, one group (n=8) fed with the SFA-CHO diet and treated with placebo and one group (n=8) fed with the SFA-CHO diet and treated with the modulator (3.0 mg kg−1). Control animals are fed a standard chow for a period of 50 days. Blood samples are collected at baseline (2 days after the reception of the animals), and 50 days after the initiation of the diet. Blood lipids are analyzed. The animals are sacrificed and necropsied.

Alternatively, the foregoing analysis comprises a plurality of groups each treated with a different dose of the compound of interest. Doses include, for example: 0.1 mg kg−1, 0.3 mg kg−1, 1.0 mg kg−1, 3.0 mg kg−1, 10 mg kg−1, 30 mg kg−1 and 100 mg kg−1. Alternatively, the foregoing analysis is carried out at a plurality of timepoints, for example, 10 weeks, 20 weeks, 30 weeks, 40 weeks, and 50 weeks.

### HDL-Cholesterol

Blood is collected in trisodium citrate (3.8%, 1:10). Plasma is obtained after centrifugation (1200 g 15 min) and immediately processed. Total cholesterol, HDL-cholesterol, and LDL-cholesterol are measured using the automatic analyzer Kodak Ektachem DT System (Eastman Kodak Company, Rochester, N.Y., USA). Samples with value parameters above the range are diluted with the solution supplied by the manufacturer and then re-analyzed. The total cholesterol/HDL-cholesterol ratio is determined. Comparison is made of the level of HDL-cholesterol between groups. Comparison is made of the total cholesterol/HDL-cholesterol ratio between groups.

Elevation of HDL-cholesterol or reduction of the total cholesterol/HDL-cholesterol ratio on administration of the compound of interest is taken as indicative of the compound having the aforesaid utility.

### Atherosclerosis

The thoracic and abdominal aortas are removed intact, opened longitudinally along the ventral surface, and fixed in neutral-buffered formalin after excision of samples from standard sites in the thoracic and abdominal aorta for histological examination and lipid composition and synthesis studies. After fixation, the whole aortas are stained with Sudan IV and pinned out flat, and digital images are obtained with a TV camera connected to a computerized image analysis system (Image Pro Plus; Media Cybernetics, Silver Spring, Md.) to determine the percentage of aortic surface involved with atherosclerotic lesions [Gerrity R G et al, Diabetes (2001) 50:1654-65; Cornhill J F et al, Arteriosclerosis, Thrombosis, and Vascular Biology (1985) 5:415-26; which disclosures are hereby incorporated by reference in their entirety]. Comparison is made between groups of the percentage of aortic surface involved with atherosclerotic lesions.

Reduction of the percentage of aortic surface involved with atherosclerotic lesions on administration of the compound of interest is taken as indicative of the compound having the aforesaid utility.

### Plasma Free Fatty Acids

It would be readily apparent to anyone of ordinary skill in the art that the foregoing in vivo pig model is easily modified in order to address, without limitation, the activity of the compound in lowering plasma free fatty acids.

### Example 8

### Assays for Determination of GPCR Activation

A variety of approaches are available for assessment of activation of human GPCRs. The following are illustrative; those of ordinary skill in the art are credited with the ability to determine those techniques that are preferentially beneficial for the needs of the artisan.

1. Membrane Binding Assays: [³⁵S]GTPγS Assay

When a G protein-coupled receptor is in its active state, either as a result of ligand binding or constitutive activation, the receptor couples to a G protein and stimulates the release of GDP and subsequent binding of GTP to the G protein. The alpha subunit of the G protein-receptor complex acts as a GTPase and slowly hydrolyzes the GTP to GDP, at which point the receptor normally is deactivated. Activated receptors continue to exchange GDP for GTP. The non-hydrolyzable GTP analog, [³⁵S]GTPγS, can be utilized to demonstrate enhanced binding of [³⁵S]GTPγS to membranes expressing activated receptors. The advantage of using [³⁵S]GTPγS binding to measure activation is that: (a) it is generically applicable to all G protein-coupled receptors; (b) it is proximal at the membrane surface making it less likely to pick-up molecules which affect the intracellular cascade.

The assay utilizes the ability of G protein coupled receptors to stimulate [³⁵S]GTPγS binding to membranes expressing the relevant receptors. The assay can, therefore, be used in the direct identification method to screen candidate compounds to endogenous GPCRs and non-endogenous, constitutively activated GPCRs. The assay is generic and has application to drug discovery at all G protein-coupled receptors.

The [³⁵S]GTPγS assay is incubated in 20 mM HEPES and between 1 and about 20 mM MgCl₂ (this amount can be adjusted for optimization of results, although 20 nM is preferred) pH 7.4, binding buffer with between about 0.3 and about 1.2 nM [³⁵S]GTPγS (this amount can be adjusted for optimization of results, although 1.2 is preferred) and 12.5 to 75 μg membrane protein (e.g, 293 cells expressing the GPR35; this amount can be adjusted for optimization) and 10 μM GDP (this amount can be changed for optimization) for 1 hour. Wheatgerm agglutinin beads (25 μl; Amersham) are then added and the mixture incubated for another 30 minutes at room temperature. The tubes are then centrifuged at 1500×g for 5 minutes at room temperature and then counted in a scintillation counter.

2. Adenylyl Cyclase

A Flash Plate™ Adenylyl Cyclase kit (New England Nuclear; Cat. No. SMP004A) designed for cell-based assays can be modified for use with crude plasma membranes. The Flash Plate wells can contain a scintillant coating which also contains a specific antibody recognizing cAMP. The cAMP generated in the wells can be quantitated by a direct competition for binding of radioactive cAMP tracer to the cAMP antibody. The following serves as a brief protocol for the measurement of changes in cAMP levels in whole cells that express a receptor.

Transfected cells are harvested approximately twenty four hours after transient transfection. Media is carefully aspirated off and discarded. 10 ml of PBS is gently added to each dish of cells followed by careful aspiration. 1 ml of Sigma cell dissociation buffer and 3 ml of PBS are added to each plate. Cells are pipetted off the plate and the cell suspension is collected into a 50 ml conical centrifuge tube. Cells are then centrifuged at room temperature at 1,100 rpm for 5 minutes. The cell pellet is carefully re-suspended into an appropriate volume of PBS (about 3 ml/plate). The cells are then counted using a hemocytometer and additional PBS is added to give the appropriate number of cells (with a final volume of about 50 μl/well).

cAMP standards and Detection Buffer (comprising 1 μCi of tracer [¹²⁵I] cAMP (50 μl) to 11 ml Detection Buffer) is prepared and maintained in accordance with the manufacturer's instructions. Assay Buffer is prepared fresh for screening and contains 50 μl of Stimulation Buffer, 3 μl of candidate compound (12 μM final assay concentration) and 50 μl cells. Assay Buffer is stored on ice until utilized. The assay, preferably carried out, for example, in a 96-well plate, is initiated by addition of 50 μl of cAMP standards to appropriate wells followed by addition of 50 μl of PBSA to wells H11 and H12. 50 μl of Stimulation Buffer is added to all wells. DMSO (or selected candidate compounds) is added to appropriate wells using a pin tool capable of dispensing 3 μl of compound solution, with a final assay concentration of 12 μM candidate compound and 100 μl total assay volume. The cells are then added to the wells and incubated for 60 minutes at room temperature. 100 μl of Detection Mix containing tracer cAMP is then added to the wells. Plates are then incubated additional 2 hours followed by counting in a Wallac MicroBeta scintillation counter. Values of cAMP/well are then extrapolated from a standard cAMP curve which is contained within each assay plate.

3. Cell-Based cAMP for Gi Coupled Target GPCRs

TSHR is a Gs coupled GPCR that causes the accumulation of cAMP upon activation. TSHR can be constitutively activated by mutating amino acid residue 623 (i.e., changing an alanine residue to an isoleucine residue). A Gi coupled receptor is expected to inhibit adenylyl cyclase, and, therefore, decrease the level of cAMP production, which can make assessment of cAMP levels challenging. An effective technique for measuring the decrease in production of cAMP as an indication of activation of a Gi coupled receptor can be accomplished by co-transfecting, non-endogenous, constitutively activated TSHR (TSHR-A623I) (or an endogenous, constitutively active Gs coupled receptor) as a “signal enhancer” with a Gi linked target GPCR to establish a baseline level of cAMP. Upon creating an endogenous or non-endogenous version of the Gi coupled receptor, the target GPCR is then co-transfected with the signal enhancer, and it is this material that can be used for screening. In some embodiments, this approach is preferably used in the direct identification of candidate compounds against Gi coupled receptors. It is noted that for a Gi coupled GPCR, when this approach is used, an inverse agonist of the target GPCR will increase the cAMP signal and an agonist will decrease the cAMP signal.

On day one, 2×10⁴ 293 cells/well are plated out. On day two, two reaction tubes are prepared (the proportions to follow for each tube are per plate): tube A is prepared by mixing 2 μg DNA of each receptor transfected into the mammalian cells, for a total of 4 μg DNA (e.g., pCMV vector; pCMV vector with mutated THSR (TSHR-A6231); TSHR-A6231 and GPCR, etc.) in 1.2 ml serum free DMEM (Irvine Scientific, Irvine, Calif.); tube B is prepared by mixing 120 μl lipofectamine (Gibco BRL) in 1.2 ml serum free DMEM. Tubes A and B are then admixed by inversions (several times), followed by incubation at room temperature for 30-45 minutes. The admixture is referred to as the “transfection mixture”. Plated 293 cells are washed with 1×PBS, followed by addition of 10 ml serum free DMEM. 2.4 ml of the transfection mixture is then added to the cells, followed by incubation for 4 hours at 37° C./5% CO₂. The transfection mixture is then removed by aspiration, followed by the addition of 25 ml of DMEM/10% Fetal Bovine Serum. Cells are then incubated at 37° C./5% CO₂. After 24 hours incubation, cells are harvested and utilized for analysis.

A Flash Plate™ Adenylyl Cyclase kit (New England Nuclear; Cat. No. SMP004A) is designed for cell-based assays, but can be modified for use with crude plasma membranes depending on the need of the skilled artisan. The Flash Plate wells contain a scintillant coating which also contains a specific antibody recognizing cAMP. The cAMP generated in the wells can be quantitated by a direct competition for binding of radioactive cAMP tracer to the cAMP antibody. The following serves as a brief protocol for the measurement of changes in cAMP levels in whole cells that express a receptor of interest.

Transfected cells are harvested approximately twenty four hours after transient transfection. Media is carefully aspirated off and discarded. 10 ml of PBS is gently added to each dish of cells followed by careful aspiration. 1 ml of Sigma cell dissociation buffer and 3 ml of PBS is added to each plate. Cells are pipetted off the plate and the cell suspension is collected into a 50 ml conical centrifuge tube. Cells are then centrifuged at room temperature at 1,100 rpm for 5 minutes. The cell pellet is carefully re-suspended into an appropriate volume of PBS (about 3 ml/plate). The cells are then counted using a hemocytometer and additional PBS is added to give the appropriate number of cells (with a final volume of about 50 μl/well).

cAMP standards and Detection Buffer (comprising 1 μCi of tracer [¹²⁵I] cAMP (50 μl) to 11 ml Detection Buffer) is prepared and maintained in accordance with the manufacturer's instructions. Assay Buffer should be prepared fresh for screening and contain 50 μl of Stimulation Buffer, 3 μl of candidate compound (12 μM final assay concentration) and 50 μl cells. Assay Buffer can be stored on ice until utilized. The assay can be initiated by addition of 50 μl of cAMP standards to appropriate wells followed by addition of 50 μl of PBSA to wells H-11 and H12. Fifty μl of Stimulation Buffer is added to all wells. Selected compounds (e.g., TSH) are added to appropriate wells using a pin tool capable of dispensing 3 μl of compound solution, with a final assay concentration of 12 μM candidate compound and 100 μl total assay volume. The cells are then added to the wells and incubated for 60 minutes at room temperature. 100 μl of Detection Mix containing tracer cAMP is then added to the wells. Plates are then incubated additional 2 hours followed by counting in a Wallac MicroBeta scintillation counter. Values of cAMP/well are extrapolated from a standard cAMP curve which is contained within each assay plate.

4. Reporter-Based Assays

a. CRE-LUC Reporter Assay (Gs-Associated Receptors)

293 or 293T cells are plated-out on 96 well plates at a density of 2×10⁴ cells per well and are transfected using Lipofectamine Reagent (BRL) the following day according to manufacturer instructions. A DNA/lipid mixture is prepared for each 6-well transfection as follows: 260 ng of plasmid DNA in 100 μl of DMEM is gently mixed with 2 μl of lipid in 100 μl of DMEM (the 260 ng of plasmid DNA consists of 200 ng of a 8xCRE-Luc reporter plasmid, 50 ng of pCMV comprising endogenous receptor or non-endogenous receptor or pCMV alone, and 10 ng of a GPRS expression plasmid (GPRS in pcDNA3 (Invitrogen)). The 8XCRE-Luc reporter plasmid is prepared as follows: vector SRIF-β-gal is obtained by cloning the rat somatostatin promoter (−71/+51) at BglV-HindIII site in the pβgal-Basic Vector (Clontech). Eight (8) copies of cAMP response element are obtained by PCR from an adenovirus template AdpCF126CCRE8 (see, Suzuki et al., Hum Gene Ther 7:1883-1893 (1996); the disclosure of which is hereby incorporated by reference in its entirety) and cloned into the SRIF-β-gal vector at the Kpn-BglV site, resulting in the 8xCRE-β-gal reporter vector. The 8xCRE-Luc reporter plasmid is generated by replacing the beta-galactosidase gene in the 8xCRE-β-gal reporter vector with the luciferase gene obtained from the pGL3-basic vector (Promega) at the HindIII-BamHI site. Following 30 minutes incubation at room temperature, the DNA/lipid mixture is diluted with 400 μl of DMEM and 100 μl of the diluted mixture is added to each well. 100 μl of DMEM with 10% FCS are added to each well after a four hour incubation in a cell culture incubator. The following day the transfected cells are changed with 200 μl/well of DMEM with 10% FCS. Eight (8) hours later, the wells are changed to 100 μl/well of DMEM without phenol red, after one wash with PBS. Luciferase activity is measured the next day using the LucLite™ reporter gene assay kit (Packard) following manufacturer instructions and read on a 1450 MicroBeta™ scintillation and luminescence counter (Wallac).

b. API Reporter Assay (Gq-Associated Receptors)

A method to detect Gq stimulation depends on the known property of Gq-dependent phospholipase C to cause the activation of genes containing AP1 elements in their promoter. A Pathdetect™ AP-1 cis-Reporting System (Stratagene, Catalogue No. 219073) can be utilized following the protocol set forth above with respect to the CREB reporter assay, except that the components of the calcium phosphate precipitate are 410 ng pAP1-Luc, 80 ng pCMV-receptor expression plasmid, and 20 ng CMV-SEAP.

c. SRF-LUC Reporter Assay (Gq-Associated Receptors)

One method to detect Gq stimulation depends on the known property of Gq-dependent phospholipase C to cause the activation of genes containing serum response factors in their promoter. A Pathdetect™ SRF-Luc-Reporting System (Stratagene) can be utilized to assay for Gq coupled activity in, for example, COS7 cells. Cells are transfected with the plasmid components of the system and the indicated expression plasmid encoding endogenous or non-endogenous GPCR using a Mammalian Transfection™ Kit (Stratagene, Catalogue #200285) according to the manufacturer's instructions. Briefly, 410 ng SRF-Luc, 80 ng pCMV-receptor expression plasmid and 20 ng CMV-SEAP (secreted alkaline phosphatase expression plasmid; alkaline phosphatase activity is measured in the media of transfected cells to control for variations in transfection efficiency between samples) are combined in a calcium phosphate precipitate as per the manufacturer's instructions. Half of the precipitate is equally distributed over 3 wells in a 96-well plate and kept on the cells in a serum free media for 24 hours. The last 5 hours the cells are incubated with, for example, 1 μM, candidate compound. Cells are then lysed and assayed for luciferase activity using a Luclite™ Kit (Packard, Cat. No. 6016911) and “Trilux 1450 Microbeta” liquid scintillation and luminescence counter (Wallac) as per the manufacturer's instructions. The data can be analyzed using GraphPad Prism™ 2.0a (GraphPad Software Inc.).

d. Intracellular IP3 Accumulation Assay (Gq-Associated Receptors)

On day 1, cells comprising the receptor of interest (endogenous or non-endogenous) can be plated onto 24 well plates, usually 1×10⁵ cells/well (although his number can be optimized). On day 2 cells can be transfected by first mixing 0.25 μg DNA in 50 μl serum free DMEM/well and 2 μl lipofectamine in 50 μl serum free DMEM/well. The solutions are gently mixed and incubated for 15-30 minutes at room temperature. Cells are washed with 0.5 ml PBS and 400 μl of serum free media is mixed with the transfection media and added to the cells. The cells are then incubated for 3-4 hours at 37° C./5% CO₂ and then the transfection media is removed and replaced with 1 ml/well of regular growth media. On day 3 the cells are labeled with ³H-myo-inositol. Briefly, the media is removed and the cells are washed with 0.5 ml PBS. Then 0.5 ml inositol-free/serum free media (GIBCO BRL) is added/well with 0.25 μCi of ³H-myo-inositol/well and the cells are incubated for 16-18 hours overnight at 37° C./5% CO₂. On Day 4 the cells are washed with 0.5 ml PBS and 0.45 ml of assay medium is added containing inositol-free/serum free media, 10 μM pargyline, 10 mM lithium chloride or 0.4 ml of assay medium and 50 μl of 10× ketanserin (ket) to final concentration of 10 μM, if using a control construct containing a serotonin receptor. The cells are then incubated for 30 minutes at 37° C. The cells are then washed with 0.5 ml PBS and 200 μl of fresh/ice cold stop solution (1M KOH; 18 mM Na-borate; 3.8 mM EDTA) is added/well. The solution is kept on ice for 5-10 minutes or until cells were lysed and then neutralized by 200 μl of fresh/ice cold neutralization sol. (7.5% HCL). The lysate is then transferred into 1.5 ml eppendorf tubes and 1 ml of chloroform/methanol (1:2) is added/tube. The solution is vortexed for 15 seconds and the upper phase is applied to a Biorad AG1-X8™ anion exchange resin (100-200 mesh). Firstly, the resin is washed with water at 1:1.25 W/V and 0.9 ml of upper phase is loaded onto the column. The column is washed with 10 mls of 5 mM myo-inositol and 10 ml of 5 mM Na-borate/60 mM Na-formate. The inositol tris phosphates are eluted into scintillation vials containing 10 ml of scintillation cocktail with 2 ml of 0.1 M formic acid/1 M ammonium formate. The columns are regenerated by washing with 10 ml of 0.1 M formic acid/3M ammonium formate and rinsed twice with dd H₂O and stored at 4° C. in water.

e. Fluorometric Imaging Plate Reader (FLIPR) Assay for the Measurement of Intracellular Calcium Concentration

Target Receptor (experimental) and pCMV (negative control) stably transfected cells from respective clonal lines are seeded into poly-D-lysine pretreated 96-well plates (Becton-Dickinson, #356640) at 5.5×10⁴ cells/well with complete culture medium (DMEM with 10% FBS, 2 mM L-glutamine, 1 mM sodium pyruvate) for assay the next day. Because the niacin receptor is Gi coupled, the cells comprising the niacin receptor can further comprise Gα15, Gα16, or the chimeric Gq/Gi alpha subunit. To prepare Fluo4-AM (Molecular Probe, #F14202) incubation buffer stock, 1 mg Fluo4-AM is dissolved in 467 μl DMSO and 467 μl Pluoronic acid (Molecular Probe, #P3000) to give a 1 mM stock solution that can be stored at −20° C. for a month. Fluo4-AM is a fluorescent calcium indicator dye.

Candidate compounds are prepared in wash buffer (1×HBSS/2.5 mM Probenicid/20 mM HEPES at pH 7.4).

At the time of assay, culture medium is removed from the wells and the cells are loaded with 100 μl of 4 μM Fluo4-AM/2.5 mM Probenicid (Sigma, #P8761)/20 mM HEPES/complete medium at pH 7.4. Incubation at 37° C./5% CO₂ is allowed to proceed for 60 minutes.

After the 1 hour incubation, the Fluo4-AM incubation buffer is removed and the cells are washed 2× with 100 μl wash buffer. In each well is left 100 μl wash buffer. The plate is returned to the incubator at 37° C./5% CO₂ for 60 minutes.

FLIPR (Fluorometric Imaging Plate Reader; Molecular Device) is programmed to add 50 μl candidate compound on the 30th second and to record transient changes in intracellular calcium concentration ([Ca2+]) evoked by the candidate compound for another 150 seconds. Total fluorescence change counts are used to determine agonist activity using the FLIPR software. The instrument software normalizes the fluorescent reading to give equivalent initial readings at zero.

Although the foregoing provides a FLIPR assay for agonist activity using stably transfected cells, a person of ordinary skill in the art would readily be able to modify the assay in order to characterize antagonist activity. Said person of ordinary skill in the art would also readily appreciate that, alternatively, transiently transfected cells could be used.

### Example 9

### Receptor Binding Assay

In addition to the methods described herein, another means for evaluating a candidate compound is by determining binding affinities to the niacin receptor. This type of assay generally requires a radiolabelled ligand to the niacin receptor.

A radiolabelled compound such as radiolabelled niacin can be used in a screening assay to identify/evaluate compounds. In general terms, a newly synthesized or identified compound (i.e., candidate compound) can be evaluated for its ability to reduce binding of the radiolabelled niacin to the niacin receptor. Accordingly, the ability to compete with the radiolabelled niacin for the binding to the niacin receptor directly correlates to the binding affinity of the candidate compound to the niacin receptor.

### Assay Protocol for Determining Receptor Binding for the Niacin Receptor:

A. Niacin Receptor Preparation

For example, HEK293 cells (human kidney, ATCC) can be transiently or stably transfected with the niacin receptor as described herein. For example, 293 cells can be transiently transfected with 10 μg human niacin receptor and 60 μl Lipofectamine (per 15-cm dish), and grown in the dish for 24 hours (75% confluency) with a media change. Cells are removed with 10 ml/dish of Hepes-EDTA buffer (20 mM Hepes+10 mM EDTA, pH 7.4). The cells are then centrifuged in a Beckman Coulter centrifuge for 20 minutes, 17,000 rpm (JA-25.50 rotor). Subsequently, the pellet is resuspended in 20 mM Hepes+1 mM EDTA, pH 7.4 and homogenized with a 50-ml Dounce homogenizer and again centrifuged. After removing the supernatant, the pellets are stored at −80° C., until used in binding assay. When used in the assay, membranes are thawed on ice for 20 minutes and then 10 mL of incubation buffer (20 mM Hepes, 1 mM MgCl₂, 100 mM NaCl, pH 7.4) is added. The membranes are then vortexed to resuspend the crude membrane pellet and homogenized with a Brinkmann PT-3100 Polytron homogenizer for 15 seconds at setting 6. The concentration of membrane protein is determined using the BRL Bradford protein assay.

B. Binding Assay

For total binding, a total volume of 50 μl of appropriately diluted membranes (diluted in assay buffer containing 50 mM Tris HCl (pH 7.4), 10 mM MgCl₂, and 1 mM EDTA; 5-50 μg protein) is added to 96-well polyproylene microtiter plates followed by addition of 100 μl of assay buffer and 50 μl of radiolabelled niacin. For nonspecific binding, 50 μl of assay buffer is added instead of 100 μl and an additional 50 μl of 10 μM cold niacin receptor is added before 50 μl of radiolabelled niacin is added. Plates are then incubated at room temperature for 60-120 minutes. The binding reaction is terminated by filtering assay plates through a Microplate Devices GF/C Unifilter filtration plate with a Brandell 96-well plate harvester followed by washing with cold 50 mM Tris HCl, pH 7.4 containing 0.9% NaCl. Then, the bottom of the filtration plates are sealed, 50 μl of Optiphase Supermix is added to each well, the top of the plates are sealed, and plates are counted in a Trilux MicroBeta scintillation counter. For compound competition studies, instead of adding 100 μl of assay buffer, 100 μl of appropriately diluted candidate compound is added to appropriate wells followed by addition of 50 μl of radiolabelled niacin.

C. Calculations

The candidate compounds are initially assayed at 1 and 0.1 μM and then at a range of concentrations chosen such that the middle dose would cause about 50% inhibition of a radiolabelled niacin binding (i.e., IC₅₀). Specific binding in the absence of candidate compound (B_{O}) is the difference of total binding (B_{T}) minus non-specific binding (NSB) and similarly specific binding (in the presence of candidate compound) (B) is the difference of displacement binding (B_{D}) minus non-specific binding (NSB). IC₅₀ is determined from an inhibition response curve, logit-log plot of % B/B_{O} vs concentration of candidate compound.

Kᵢ is calculated by the Cheng and Prustoff transformation:

Kᵢ=IC₅₀/(1+[L]/K_{D})

where [L] is the concentration of a radiolabelled niacin used in the assay and K_{D} is the dissociation constant of a radiolabelled niacin determined independently under the same binding conditions.

Applicants reserve the right to exclude any one or more compounds from any of the embodiments of the invention. Applicants also reserve the right to exclude, for example, any formulation or amount of niacin, a niacin analog or niacin receptor agonist, any niacin receptor partial agonist, or any combination therapy.

Throughout this application, various publications, patents and published patent applications are cited. The disclosures of these publications, patents and published patent applications referenced in this application are hereby incorporated by reference in their entirety into the present disclosure. Citation herein by Applicant of a publication, patent, or published patent application is not an admission by Applicant of said publication, patent, or published patent application as prior art.

Modifications and extension of the disclosed inventions that are within the purview of the skilled artisan are encompassed within the above disclosure and the claims that follow.

What is claimed is:

1. A method of identifying a niacin receptor modulator with reduced flushing effect compared to niacin or a niacin analog, comprising determining the MAP kinase activity of said modulator, wherein a decrease in MAP kinase activity induced by said modulator compared to MAP kinase activity induced by niacin or a niacin analog indicates that said modulator has reduced flushing effect when compared to niacin or a niacin analog.

2. A method of identifying a niacin receptor modulator with reduced flushing effect compared to niacin, comprising determining the MAP kinase activity of said modulator, wherein a decrease in MAP kinase activity induced by said modulator compared to MAP kinase activity induced by niacin indicates that said modulator has reduced flushing effect when compared to niacin.

3. A method of identifying a niacin receptor modulator with reduced flushing effect compared to niacin, comprising:
a) identifying a niacin receptor modulator, and
b) determining the MAP kinase activity of said modulator,
wherein a decrease in MAP kinase activity induced by said modulator compared to MAP kinase activity induced by niacin indicates that said modulator has reduced flushing effect when compared to niacin.

4. The method of claim 1, 2 or 3, wherein said decrease in MAP kinase activity induced by said modulator is at least two standard deviations below the level of MAP kinase activity induced by niacin.

5. The method of claim 1, 2 or 3, wherein an antibody based assay is used to determine said MAP kinase activity.

6. The method of claim 1, 2 or 3, wherein an ELISA is used to determine said MAP kinase activity.

7. A niacin receptor modulator with reduced flushing effect compared to niacin or a niacin analog, wherein said modulator is identified as a modulator with reduced flushing effect compared to niacin or a niacin analog according to the method of claim 1.

8. The modulator of claim 1, 2, 3 or 7, wherein said modulator is a niacin receptor agonist or partial agonist.

9. The modulator of claim 1, 2, 3 or 7, wherein said modulator is an anti-lipolytic compound.

10. The modulator of claim 1, 2, 3 or 7, wherein said modulator has no significant flushing effect when compared to niacin or a niacin analog.

11. A method for preparing a composition which comprises identifying a niacin receptor modulator with reduced flushing effect compared to niacin or a niacin analog and then admixing said modulator with a carrier, wherein said modulator is identified by the method of claim 1.

12. A pharmaceutical composition comprising, consisting essentially of, or consisting of the modulator of claim 7.

13. A method for preventing or treating a lipid-associated disorder in a subject, comprising administering to said subject an effective lipid altering amount of a niacin receptor modulator with reduced flushing effect compared to niacin or a niacin analog, identified by the method of claim 1.

14. The method of claim 13, wherein said lipid-associated disorder is dyslipidemia, hyperlipidemia, hypertriglyceridemia, hypercholesterolemia, atherosclerosis, metabolic syndrome, heart disease, stroke, or peripheral vascular disease.

15. The method of claim 13, wherein said lipid-associated disorder is dyslipidemia.

16. The method of claim 13, wherein said lipid-associated disorder is atherosclerosis.

17. The method of claim 13, further comprising administering to said subject an effective amount of an agent used for the treatment of obesity or diabetes in combination with an effective amount of niacin receptor modulator with reduced flushing effect compared to niacin or a niacin analog identified by the method of claim 1.

18. The method of claim 13, wherein the subject is a mammal.

19. The method of claim 13, wherein the subject is a human.

20. A method for decreasing LDL levels in a subject in need thereof, comprising administering to said subject an effective amount of the modulator of claim 7.

21. A method for decreasing triglyceride levels in a subject in need thereof, comprising administering to said subject an effective amount of the modulator of claim 7.

22. A method for increasing HDL levels in a subject in need thereof, comprising administering to said subject an effective amount of the modulator of claim 7.

23. A method for the manufacture of a medicament comprising the modulator of claim 7, for use as a lipid altering agent.

24. A method for the manufacture of a medicament comprising the modulator of claim 7, for use in the treatment of a lipid-associated disorder.


SEQUENCE LISTING (2 sequences)

SEQ ID NO: 1 - DNA, 1092 bp, Homo sapien
atgaatcggcaccatctgcaggatcactttctggaaatagacaagaagaactgctgtgtgttccgagatgacttcattgtcaaggtgttgccgccggtgttggggctggagtttatcttcgggcttctgggcaatggccttgccctgtggattttctgtttccacctcaagtcctggaaatccagccggattttcctgttcaacctggcagtggctgactttctactgatcatctgcctgcccttcctgatggacaactatgtgaggcgttgggactggaagtttggggacatcccttgccggctgatgctcttcatgttggctatgaaccgccagggcagcatcatcttcctcacggtggtggcggtagacaggtatttccgggtggtccatccccaccacgccctgaacaagatctccaatcggacagcagccatcatctcttgccttctgtggggcatcactattggcctgacagtccacctcctgaagaagaagatgccgatccagaatggcggtgcaaatttgtgcagcagcttcagcatctgccataccttccagtggcacgaagccatgttcctcctggagttcttcctgcccctgggcatcatcctgttctgctcagccagaattatctggagcctgcggcagagacaaatggaccggcatgccaagatcaagagagccatcaccttcatcatggtggtggccatcgtctttgtcatctgcttccttcccagcgtggttgtgcggatccgcatcttctggctcctgcacacttcgggcacgcagaattgtgaagtgtaccgctcggtggacctggcgttctttatcactctcagcttcacctacatgaacagcatgctggaccccgtggtgtactacttctccagcccatcctttcccaacttcttctccactttgatcaaccgctgcctccagaggaagatgacaggtgagccagataataaccgcagcacgagcgtcgagctcacaggggaccccaacaaaaccagaggcgctccagaggcgttaatggccaactccggtgagccatggagcccctcttatctgggcccaacctctccttaa

SEQ ID NO: 2 - protein, 363 aa, Homo sapien [sequence omitted]